(19) United States
(12) Patent Application Publication
(10) Pub. No.: US 2024/0296914 A1
(43) Pub. Date: Sep. 5, 2024

(54) UV-VIS SPECTRA PREDICTION

(71) Applicant: Collaborations Pharmaceuticals, Inc., Fuquay Varina, NC (US)

(72) Inventors: Sean Ekins, Fuquay Varina, NC (US); Kushal Batra, Raleigh, NC (US); Maggie Anne Hupcey, Fuquay Varina, NC (US); Fabio Lee Urbina, Chapel Hill, NC (US)

(73) Assignee: Collaborations Pharmaceuticals, Inc., Fuquay Varina, NC (US)

(21) Appl. No.: 18/272,982

(22) PCT Filed: Jan. 18, 2022
(86) PCT No.: PCT/US2022/012783

§ 371 Date: Jul. 18, 2023

(30) Foreign Application Priority Data
Jan. 18, 2021 (IN) 202121002304

(51) Int. Cl. G16C 20/30; G16C 20/20; G16C 20/70

(52) U.S. Cl. CPC G16C 20/30; G16C 20/20; G16C 20/70

(57) ABSTRACT

Methods, systems, and computer readable media for predicting a spectrum, e.g., a UV-Vis spectrum, of a target molecule are disclosed. According to one aspect, a descriptor, such as a SMILES sequence or extended connectivity fingerprint, of the target molecule can be provided or generated and analyzed via a trained machine learning model, such as a trained long-short term memory (LSTM) model, to predict the spectrum of the target molecule. As determined via a variety of statistical measures, the methods, systems and computer readable media can predict the complete UV spectrum of a target molecule with high accuracy over the entire spectrum.

### STATEMENT OF GOVERNMENT INTEREST

This invention was made with government support under Contract No. HR0011-19-C-O108 awarded by the Defense Advanced Research Projects Agency (DARPA) and Award Nos. R43ES031038 and R44GM122196-02A1 from the National Institutes of Health. The government has certain rights in the invention.

### RELATED APPLICATIONS

The presently disclosed subject matter claims the benefit of Indian Patent Application No. 202121002304, filed Jan. 18, 2021, the disclosure of which is incorporated herein by reference in its entirety.

### TECHNICAL FIELD

The presently disclosed subject matter relates to systems, methods, and computer readable media for predicting a spectrum of a target molecule, e.g., an ultraviolet-visible (UV-Vis) spectrum of a target molecule. The systems, methods, and media involve the use of machine learning models, such as a long-short term memory model, that can be trained with training data sets. The methods, systems, and media can be used, for example, in methods of detecting a target molecule in a mixture of molecules or in predicting phototoxicity.

### Abbreviations

%=percent or percentage
° C.=degrees Celsius
λₘₐₓ=maximum wavelength absorption band
μL=microliter
μM=micromolar
2D=two dimensional
3D=three dimensional
DAD=diode array detector
DFT=density functional theory
DMSO dimethyl sulfoxide
DTW dynamic time warping
ECFP=extended connectivity fingerprint
ECFP6=extended connectivity fingerprint diameter 6
FWHM=full width at half maximum
HPLC=high-performance liquid chromatography
IR=infrared
LSTM=long-short term memory
MAE=mean absolute error
min=minutes
mM=millimolar
MS=mass spectroscopy
nm=nanometer
NMR=nuclear magnetic resonance
PDA=photodiode array
RMSD=root-mean-squared deviation
RMSE=root mean square error
SEQ2SEQ=sequence-to-sequence
SMILES=simplified molecular-input line-entry system
TD-DFT=time dependent-density functional theory
t-SNE=t-distributed stochastic neighbor
UHPLC=ultra-high-performance liquid chromatography
UV-Vis=ultraviolet-visible
XIC=extracted ion chromatography

### BACKGROUND

Ultraviolet-visible (UV-Vis) spectroscopy is an analytical technique used with ions, small molecules, and large macromolecules. The spectra generated can be used to determine, identify, and quantify molecules depending on the solvent, pH, and other factors. For example, in organic chemistry, reaction products are frequently analyzed using high-performance liquid chromatography (HPLC) in combination with ultraviolet (UV) or UV-Vis spectroscopy to identify chemical reaction products from complex mixtures, e.g., generated by automated molecular design, synthesis and analytical cycles. However, in many cases, if the molecule has never been synthesized before, there will be no spectra for comparison. Additionally, dye research for biotechnology, genomics, immunoassays, and drug discovery utilizes different fluorophores and makes frequent use of the UV-Vis absorbance spectra. The UV spectrum for a molecule is also useful for predicting the phototoxicity of a molecule, a property that can be of relevance to drug discovery. Thus, the ability to accurately predict a UV spectrum at the earliest stages of drug discovery, before compound synthesis, would be beneficial to the field. Density functional theory (DFT) calculations are often used to predict such spectra for molecules and aid in their design. However, these types of quantum chemistry approaches have been developed for decades with only modest success as measured by the root-mean-squared deviation (RMSD).

Accordingly, there is an ongoing need for additional methods and systems for predicting spectra, e.g., UV spectra, of chemical compounds, particularly those that do not resort to quantum chemistry approaches and that are rapid, efficient, and accurate.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a system for predicting a spectrum of a target molecule, the system comprising: one or more processors and a memory communicably coupled to the one or more processors and storing: a first module comprising instructions that when executed by the one or more processors cause the one or more processors to receive or generate a descriptor of the target molecule; and a second module including instructions that when executed by the one or more processors cause the one or more processors to apply a trained machine learning model to the descriptor of the target molecule to predict a spectrum of the target molecule, and further wherein the second module includes instructions to provide the predicted spectrum as an electronic output.

In some embodiments, the descriptor of the target molecule is a simplified molecular-input line-entry system (SMILES) sequence, a tokenized SMILES sequence, or an extended connectivity fingerprint (ECFP). In some embodiments, the descriptor of the target molecule is an ECFP and the first module comprises instructions that when executed by the one or more processors cause the one or more processors to divide the ECFP into a plurality of groups, optionally 8 groups, and to convert each of the groups into a decimal value for input into the trained machine learning model. In some embodiments, the descriptor of the target molecule is a tokenized SMILES sequence and the second module further comprises instructions that when executed by the one or more processors cause the one or more processors to generate a vector of weights for each character of the tokenized SMILES sequence at one or more wavelength values.

In some embodiments, the trained machine learning model is a trained model for time series data prediction and/or a trained long-short term memory (LSTM) model or a machine learning model similar thereto. In some embodiments, the predicted spectrum is an ultraviolet-visible (UV-Vis) spectrum.

In some embodiments, the system is further configured to receive data related to an observed spectrum of the target molecule and the second module further comprises instructions for comparing the predicted spectrum with the observed spectrum, optionally using Dynamic Time Warping (DTW). In some embodiments, the second module further comprises instructions for measuring the root-mean-squared deviation (RMSD) of the predicted spectrum.

In some embodiments, the system is further configured to generate the trained machine learning model by: acquiring or generating training data, wherein said training data comprises (a) a plurality of observed spectra, wherein each of the plurality of observed spectra is the observed spectra for a different training molecule in a training set comprising a plurality of training molecules; and (b) a plurality of descriptors, wherein each of the plurality of descriptors is a descriptor for a different training molecule in the training set; and training a machine learning model using the training data, thereby generating the trained machine learning model.

In some embodiments, the presently disclosed subject matter provides a method for predicting a spectrum of a target molecule, optionally a UV-Vis spectrum, comprising: (i) receiving and/or generating a descriptor of the target molecule; and (ii) applying a trained machine learning model to the descriptor of the target molecule with at least one processor to provide a predicted spectrum of the target molecule. In some embodiments, the descriptor of the target molecule is a simplified molecular-input line-entry system (SMILES) sequence, a tokenized SMILES sequence, or an extended connectivity fingerprint (ECFP).

In some embodiments, the receiving and/or generating data defining the descriptor of the target molecule of step (i) comprises generating an ECFP of the target molecule and further comprises dividing the ECFP into a plurality of groups, optionally 8 groups, and converting each group into a decimal value. In some embodiments, the descriptor of the target molecule is a tokenized SMILES sequence and the applying of step (ii) comprises generating a vector of weights for each character in the tokenized SMILES sequence at one or more wavelength values.

In some embodiments, the trained machine learning model is a trained machine learning model for time series data prediction and/or a trained long-short term memory (LSTM) model or a machine learning model similar thereto. In some embodiments, the method further comprises comparing the predicted spectrum with an experimentally observed spectrum, optionally using Dynamic Time Warping (DTW). In some embodiments, the method further comprises analyzing the predicted spectrum to predict phototoxicity of the target molecule. In some embodiments, the target molecule is a potential dye or colorant molecule and the predicted spectrum is a visible spectrum that provides information regarding color of the target molecule.

In some embodiments, the method further comprises, prior to step (ii), generating the trained machine learning model, wherein generating the trained machine learning model comprises: acquiring or generating training data, wherein said training data comprises (a) a plurality of observed spectra, wherein each of the plurality of observed spectra is the observed spectra for a different training molecule in a training set comprising a plurality of training molecules; and (b) a plurality of descriptors, wherein each of the plurality of descriptors is a descriptor for a different training molecule in the training set; and training a machine learning model using the training data, thereby generating the trained machine learning model.

In some embodiments, the presently disclosed subject matter provides a method for detecting a target molecule in a mixture of molecules, wherein the method comprises: (a) obtaining a spectrum of the mixture of molecules, optionally wherein the spectrum is a UV-Vis spectrum; and (b) comparing the spectrum from (a) with a predicted spectrum of the target molecule, optionally a predicted UV-Vis spectrum of the target molecule, wherein said predicted spectrum is obtained by performing a method of predicting a spectrum or a target molecule and/or using a system as described herein. In some embodiments, the spectrum obtained in step (a) is a spectrum obtained from an aliquot of a chromatography eluant, optionally of a synthetic reaction mixture, further optionally wherein the chromatography eluant is a high-performance liquid chromatography (HPLC) eluant. In some embodiments, the spectrum obtained in step (a) is a spectrum obtained from a sample present in a well of a microarray or microwell plate, optionally wherein said microarray or microwell plate comprises a plurality of wells and wherein each of the plurality of wells contains a sample that is different from the sample present in any other of the plurality of wells.

In some embodiments, the presently disclosed subject matter provides a method for detecting a target molecule in a mixture of molecules, wherein the method comprises: (a) providing a microarray or microwell plate comprising a plurality of wells, wherein each of the plurality of wells contains a sample that comprises one or more molecules, and wherein each of the plurality of wells contains a sample that comprises a different molecule or combination of molecules than in a sample present in any other of the plurality of wells; (b) obtaining a spectrum, optionally a UV-Vis spectrum, of the sample present in each of a plurality of wells of the microarray or microwell plate, thereby obtaining a plurality of spectra; and (c) comparing the spectra from (b) with a predicted spectrum of the target molecule, optionally a predicted UV-Vis spectrum of the target molecule, wherein said predicted spectrum is obtained by performing a method of predicting a spectrum or a target molecule and/or using a system as described herein.

In some embodiments, the presently disclosed subject matter provides a non-transitory computer readable medium comprising computer executable instructions embodied in a computer readable medium that when executed by a processor of a computer control the computer to perform steps comprising: receiving and/or generating a descriptor of the target molecule, optionally a simplified molecular-input line-entry system (SMILES) sequence, a tokenized SMILES sequence, or an extended connectivity fingerprint of the target molecule; and applying a trained machine learning model to the descriptor to predict a spectrum of the target molecule.

Accordingly, it is an object of the presently disclosed subject matter to provide systems for predicting a spectrum of a target molecule, as well as related methods and non-transitory computer readable media.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIGS. 1A and 1B are a pair of graphs showing the actual (black line) and predicted (grey line) ultraviolet-visible (UV-Vis) spectra (absorbance in arbitrary units versus wavelength in nanometers (nm)) for two exemplary compounds from a model test set with 288 molecules described in Example 1. The average root-mean-squared deviation (RMSD) for the compound in FIG. 1A was 0.05. The average RMSD for the compound in FIG. 1B is 0.08.

FIGS. 2A-2C are a set of three graphs showing compound test set correlation between dynamic time warping (DTW) and root-mean-squared deviation (RMSD) for (FIG. 2A) a 290 compound test set and (FIGS. 2B and 2C) a 298 compound test set. FIG. 2C is an expanded version of the portion of the graph shown in FIG. 2B between RMSD 0 and 1. The red line in each of the graphs indicates a linear fit.

FIGS. 3A-3C: FIG. 3A is a schematic diagram showing an overview of the experimental workflows for generating data with a photodiode array (PDA) or plate reader and the datasets described in Example 2. FIG. 3B is a t-distributed stochastic neighbor embedding (t-SNE) plot of chemical structure overlap between compounds generated by HPLC (Dataset I) and spectrophotometer (Dataset II). Compounds that are structurally similar are close together in 2D space. No compounds are duplicated between the two datasets. FIG. 3C is a schematic diagram showing two long-short term memory (LSTM) architectures used for spectrum prediction. On the left-hand side is a LSTM model composed of LSTM layers followed by dense layers for the output, which takes in a simplified molecular-input line-entry system (SMILES) string or extended connectivity fingerprint diameter 6 (ECFP6) as input. On the right-hand side of FIG. 3C is a diagram of architecture of a Seq2Seq model with attention, which uses bi-directional LSTMs for the encoder and Luong attention and takes in SMILES strings as input.

FIG. 4 is a graph (average root mean square error (RMSE) versus number of layers) showing parameter optimization for the number of layers in the long-short term memory (LSTM) model.

FIGS. 5A and 5B: FIG. 5A is a schematic drawing showing the chemical structures of representative molecules from Dataset I. FIG. 5B is a series of graphs showing the comparison of different descriptors to predict UV spectra for different representative molecules from Dataset I.

FIGS. 6A and 6B are graphs showing the average silhouette method applied to (FIG. 6A) Dataset I of Example 2 and (FIG. 6B) Dataset II of Example 2 to determine the optimal number of clusters (k). Comparison of FIGS. 6A and 6B suggests that there is an increased number of clusters for smaller Dataset I.

FIGS. 7A and 7B are a pair of graphs showing (FIG. 7A) a 150 compound test set correlation between dynamic time warping (DTW) and root mean square error (RMSE) for Dataset I of Example 2 and (FIG. 7B) a 333 compound test set correlation between DTW and RMSE for Dataset II of Example 2. The straight line indicates linear fit

FIGS. 8A-8D are a series of t-distributed stochastic neighbor embedding (t-SNE) plots. FIGS. 8A and 8B are t-SNE plots of overlap between the test and training sets of Dataset I (FIG. 8A) and t-SNE of the test set only, shaded according to Mean Absolute Error (FIG. 8B). FIGS. 8C and 8D are t-SNE plots of overlap between the test and training sets of plate-derived compounds (FIG. 8C) and t-SNE of the test set only, shaded according to Mean Absolute Error (FIG. 8D).

FIG. 9 is a t-distributed stochastic neighbor embedding (t-SNE) plot of overlap between 17 additional test compounds in Dataset III and the model from Dataset I described in Example 2.

FIGS. 10A-10C show the exploration of the sequence-to-sequence (SEQ2SEQ) model's attention weights. FIG. 10A is a schematic diagram showing the encoder side of Seq2Seq and the generation of an attention weight vector for each tokenized simplified molecular-input line-entry system (SMILES) input. FIG. 10B is a graph of example spectra and selected wavelengths at which the attention weights are visualized. FIG. 10C is a series of graphs of attention weights for each token SMILES input for each of the four chose wavelengths. At each prediction step, the attention weights focus on the most relevant SMILES input token as represented by the weight value.

FIG. 11 is a graph showing the actual and predicted ultraviolet-visible (UV-Vis) spectra for an exemplary compound (SRI-1053288-001) using the model derived from Dataset I of Example 2.

FIG. 12 is a schematic diagram of a system according to the presently disclosed subject matter.

FIGS. 13A-13C are schematic diagrams of (FIG. 13A) a method according to the presently disclosed subject matter for predicting a spectrum of a target molecule, (FIG. 13B) the method of FIG. 13A further comprising optional additional steps for predicting additional properties or comparing the predicted spectrum with an observed spectrum of the target molecule; and (FIG. 13C) a method of the presently disclosed subject matter comprising a training phase for generating a trained model and a predicting phase for generating a predicted spectrum.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of size, temperature, time, weight, volume, concentration, capacitance, specific capacity, discharge capacity, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g. 1 to 5 includes, but is not limited to, 1, 1.5, 2, 2.75, 3, 3.90, 4, and 5).

The term “module” as used herein refers to software in combination with hardware and/or firmware for implementing features described herein. In some embodiments, a module can include a field-programmable gateway array (FPGA), an application-specific integrated circuit (ASIC), or a processor.

### II. General Considerations

Molecules absorb ultraviolet (UV) and visible (Vis) light with the excitation of their electrons to higher-energy molecular orbitals. The intensity of absorption varies as a function of wavelength, with greatest absorption corresponding to wavelengths having the energies of allowed electronic transitions. This variation, the absorption spectrum,¹⁷ underpins UV-Vis spectroscopy, a commonly used analytical technique to quantitatively determine different analytes, such as solutions of macromolecules, conjugated organic compounds and transition metal ions.¹⁸

Because the UV-Vis spectrum of a compound is sensitive to its structure, UV-Vis spectroscopy can be used to identify molecules with reliability comparable to that of low-resolution tandem mass spectrometry.¹ Thus, UV-Vis spectroscopy is useful as a rapid, inexpensive, and non-destructive confirmatory tool in chemical synthesis and purification and in natural product isolation. In organic chemistry, reaction products are frequently analyzed using high-performance liquid chromatography (HPLC) coupled with UV spectrophotometry. For instance, routine analysis of reaction mixtures by HPLC often involves a photodiode array (PDA) detector that measures the UV-Vis spectra continuously during a chromatographic separation. UV-Vis spectroscopy is also used to monitor chemical reactions in situ, such as in flow reactors.¹⁹ The spectra generated can also be used to quantify the amount of compound using Beer-Lambert law. However, identification of a compound from its UV-Vis spectrum typically relies on comparison to an experimentally observed reference spectrum. Being able to identify molecules from complex mixtures using an accurate and readily available predicted spectrum would accelerate HPLC analysis of reaction products with value in automated molecular design, synthesis and analytical cycles.

Dye research for biotechnology, genomics, immunoassays, and drug discovery utilizes different fluorophores and makes frequent use of the UV-Vis absorbance spectra for molecules. A predicted spectrum for novel dyes would accelerate this process with value in automated molecular design and synthesis and analytical research. Generating spectra for a wide variety of molecules could also be useful in training recurrent neural network models in order to de novo design molecules with a particular spectra of interest for specific applications in developing dyes or reagents with ideal physicochemical properties.

The UV spectrum for a molecule is also useful for predicting other optical and chemical properties, such as phototoxicity, which is particularly relevant to drug discovery and is evaluated prior to Phase III clinical trials.¹ The ability to accurately predict a UV spectrum at the earliest stages of drug discovery, before compound synthesis, would be highly beneficial to the field and also cost effective, for example, compared to embarking on research and development efforts for a compound that can later be identified with this liability. Recent efforts have compiled data on compounds known to be phototoxic in in vitro assays, and this has been used for machine learning with quantum chemical descriptors producing accuracies between 83-85%.¹ Predicting the UV-Vis spectrum of a compound before synthesis and experimental testing also offers advantages in terms of avoiding molecules that interfere with high throughput assays.²⁰

Ab initio time dependent-density functional theory (TD-DFT) calculations are often used to predict electronic absorption spectra¹⁷ and the maximum wavelength absorption bands (λmax) for molecules to aid in their design for numerous applications.¹⁻⁹ However, these types of quantum chemistry approaches have been developed for decades with only modest success as measured by the root-mean-squared deviation (RMSD). Hence, prior to the presently disclosed subject matter, predicting the UV spectrum of a molecule of interest was still an unsolved problem in chemistry in terms of efficiency and with high accuracy. Alternative approaches to predicting a UV-Vis spectrum from a molecule structure alone, without resorting to quantum chemistry approaches, would offer a quicker, and potentially more informative, route for large collections of molecules.

One challenge in the application of machine learning to the prediction of UV-Vis absorption spectra is the paucity of available training data. There are few open-source databases of UV-Vis absorption spectra, and most focus only on Amax rather than the full spectrum within a useful wavelength range. The currently available databases with UV-Vis spectra include the Max Weaver dye library,²² NIST Chemistry Webbook,²³ PhotochemCAD,²⁴ UV/Vis+ photochemistry database²⁵ and the DSSC Database²⁶ ranging from hundreds to several thousand molecules.²⁷ Few of these databases provide full spectra for use in machine learning, and most are biased toward specific classes of molecular structures, particularly dyes. PhotochemCAD provides spectra of ˜339 entries for download; however, the wavelength range over which the spectra are measured varies, making compilation for machine learning purposes difficult.²⁴ Commercial UV-Vis spectral databases include the UV-Vis spectral database collection available under the trade name KNOWITALL® from John Wiley & Sons, Inc. (Hoboken, New Jersey, United States of America), having over 30,000 spectra, with over 60% of them covering a narrow range 200-350 nm.²⁸

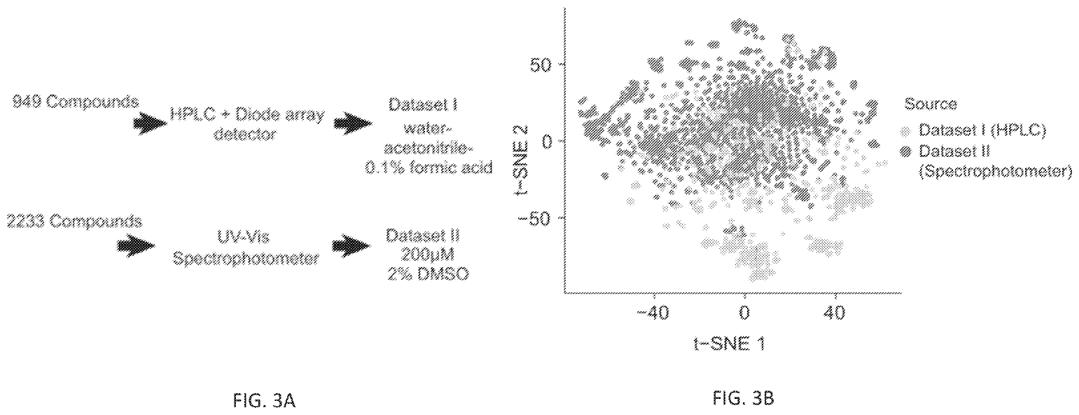

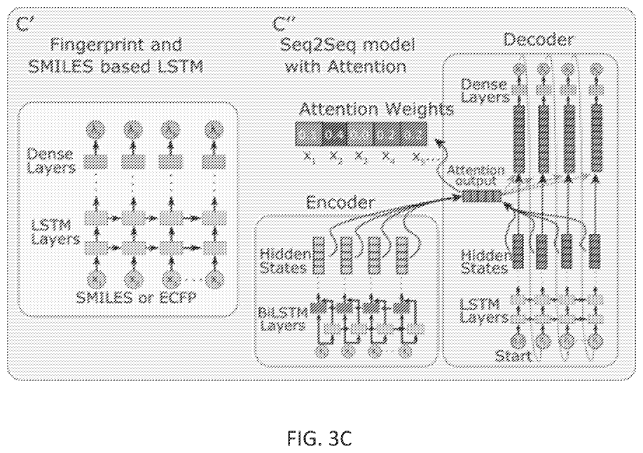

According to one aspect of the presently disclosed subject matter is the use of novel datasets of spectra for a diverse collection of small molecules. See FIG. 3A and Datasets I and II of Example 2, described hereinbelow. As described herein, these data have been used with multiple machine learning approaches to reliably predict spectra for new molecules. Multiple measures were used to compare predicted to experimental spectra, including root mean square error (RMSE), R², mean absolute error (MAE), RMSE of derivative spectra, and dynamic time warping (DTW), which is a distance measure technique that allows a non-linear mapping between two signals by minimizing the distance between them.¹³ Altogether, the presently disclosed approach, also referred to herein as the UV-ADVISOR™ approach, does not involve time-intensive quantum chemistry calculations and provides accurate, multiple-wavelength spectrum predictions (across a complete spectrum rather than just the λₘₐₓ), comparable to or better than currently used models.

### III. Systems, Methods and Computer Readable Media

The presently disclosed subject matter provides, in some embodiments, systems, methods and non-transitory, computer readable media to predict a spectrum (e.g., a UV-Vis spectrum) of a target molecule from the molecule's structure alone, without resorting to quantum chemistry approaches. The presently disclosed methods, systems and media provide a quicker route to generate spectral data for very large databases of molecules. In some embodiments, the presently disclosed subject matter is fast and predictive for spectra with low RMSD˜0.2. In some embodiments, the presently disclosed subject matter further comprises methods of comparing spectra (e.g., UV-Vis spectra), which can also aid in determining the quality of the predictions.

Previously, using a machine learning approach for UV-Vis spectra prediction would have been expected to require a very large training dataset of diverse spectra to cover chemistry space. However, according to the presently disclosed subject matter, relatively small databases (e.g., databases comprising 1000 molecules or fewer) can result in reliable spectrum predictions. In some embodiments, observed and predicted spectra can be compared using RMSE, R², MAE, RMSE of derivative spectra, and/or DTW. In some embodiments, observed and predicted spectra can be compared using DTW

In some embodiments, the presently disclosed systems, methods, and media make use of a Deep Learning Machine Learning model algorithm called LSTM (Long-Short Term Memory) model. This model has mainly been used for time series data prediction. According to the presently disclosed subject matter, spectra prediction (e.g., UV-Vis spectra prediction) can be considered as a time series with lambda as the x-axis as it is proportional to time linearly (using Wavelength(lambda)=c(speed of light)*t(time)). In some embodiments, a wavelength window of from about 220 nm to about 400 nm is used. In some embodiments, an extended connectivity fingerprint (ECFP) (e.g., ECFP6, also known as Morgan Fingerprint with diameter 3) for each molecule calculated from the SMILES (using RDKit library in python) is employed. This fingerprint array is composed of binary bits 1 and 0 and the array's total size set is 2048 bits (default value). In some embodiments, these 2048 bits are divided into groups of 256 bits, resulting in a total of 8 groups for a molecule (8-bit). These groups are then converted to base 10 integers (i.e., to decimal values). The LSTM model takes these 8 decimal values as parameters along with the wavelength value (generated in 1 nm increments for a spectra) for each molecule to build a model. The test column is the absorbance value to be predicted. Typically, the model is trained using a 70:15:15 (train: test: validation) split. These splits are identified on the basis of clustering based on molecular structural similarity using MDL descriptors. The LSTM model has 4 layers with return sequence set to True and activation function=‘relu’; and 3 Dense layers with a final layer being the output layer. The model makes use of Adam Optimizer and loss is measured using the Mean Square Error while training. The number of epochs the code runs for is 2500 with batch size=5. For each molecule, the RMSD and the DTW are calculated using the scikit library in python. A training set of >154 molecules and test set of 58 molecules resulted in an average RMSD of ˜0.31. On a larger training set of 683 and a test set of 291 molecules, the RMSD for the test set was 0.18. The DTW, a second metric, provides a more interpretable method to compare predicted and observed spectra to access model quality. The RMSD values observed using the presently disclosed systems, methods, and media were comparable or better than those achieved by DFT with the advantage that once the model is rapidly trained (in a fraction of the time compared with DFT), the spectra can be rapidly predicted for input molecules. Thus, according to the presently disclosed subject matter, libraries of millions to billions of molecules can be scored, making it possible to more rapidly and reliably identify molecules with a desirable spectrum.

In some embodiments, a model is used that is based on encoder-decoder acrhitectures^{31,32} with an attention mechanism for language translation. This model is based on approaching spectrum prediction as a sequence to sequence (Seq2Seq) translation problem between a chemical structure (represented by SMILES string) and a wavelength sequence output. Each unique character in the SMILES vocabulary was represented as a separate integer, except for Br and any closed brackets notation, which were given their own separate integers. A beginning (<B>) token was added in the front of each SMILES, and an end-of-sequence token (<EOS>) was added at the end of each SMILES string, each with their own unique integer representation. Each SMILES string was thus tokenized by conversion into an integer representation which was used as the input sequence into the spectra models. The test column is the absorbance value to be predicted. The Seq2Seq model was trained using a randomized 70:15:15 (train: test: validation) split.

The presently disclosed systems and methods can have applications for new dye discovery, organic chemistry reaction monitoring, drug discovery, and phototoxicity prediction. In some embodiments, the predicted spectra can be compared to spectra of samples obtained during chromatography (e.g., HPLC) of mixtures, e.g., reaction mixtures. In some embodiments, the predicted spectra can be compared to spectra obtained from microwell plates comprising arrays of different molecules or mixtures of molecules. The presently disclosed subject matter is also believed to be applicable to the prediction of spectra related to types of spectroscopy other than UV-Vis spectroscopy, such as, spectroscopy involving the absorption and/or reflectance of other types of electromagnetic radiation, including, but not limited to infrared (IR) spectroscopy.

In some embodiments, the presently disclosed subject matter provides a system for predicting a spectrum of a target molecule. In some embodiments, the target molecule is a potential dye molecule, a potential intermediate, side-product, or product of an organic chemistry reaction (e.g., a functional group transformation), a potential small molecule drug, or a potential drug metabolite. In some embodiments, the target molecule is a molecule having a molecular weight of about 1000 daltons or less, about 900 daltons or less, about 800 daltons or less, or about 750 daltons or less. The molecule can be aliphatic or aromatic or include combinations of aliphatic and aromatic groups. The target molecule can include one or more chemical functional groups, such as, but not limited to, alkyl, alkenyl, alkynyl, cycloalkyl, aryl, heteroaryl, heterocyclic, keto, halo, hydroxyl, amino, thio, cyano, nitro, azo, carboxylic acid, ester, amide, carbamate, etc. In some embodiments, the target molecule comprises a chromophore, i.e., a group or groups that absorb visible light. In some embodiments, the target molecule can include one or more metal ions (e.g., one or more transition metal ions), for example as part of a coordination complex. In some embodiments, the target molecule is a molecule having a molecular weight larger than about 1000 daltons. Such larger target molecules can include, but are not limited to, larger natural products and biopolymers, such as polyketides (e.g., macrolides) and peptides or proteins with a molecular weight larger than about 1000 daltons.

In some embodiments, the system comprises one or more processors and a memory communicably coupled to the one or more processors and storing: a first module comprising instructions that when executed by the one or more processors cause the one or more processors to receive and/or generate a descriptor of a target molecule; and a second module including instructions that when executed by the one or more processors cause the one or more processors to apply a trained machine learning model to the descriptor of the target molecule to predict a spectrum of the target molecule and instructions to provide the predicted spectrum as an electronic output. The trained machine learning model can be a machine learning model trained to correlate descriptors to spectral information (e.g., multi-wavelength UV-Vis absorption data) using data from a training set comprising a plurality of different training molecules. The data from the training set of molecules can include observed spectra (e.g., UV-Vis spectra, either experimentally observed or previously experimentally generated and retrieved from a database) of a plurality of the training molecules and descriptors of a plurality of the training molecules.

As used herein, the term “descriptor” refers to chemical fingerprints and/or structural or physicochemical molecular descriptors including, but not limited to computer readable forms for chemical structures (e.g., InCHI, SDF, or SMILES). Thus, the term “descriptor” as used herein can refer to a computer readable form for a structural or physicochemical property of a molecule or part of a molecule. Molecular descriptors can include descriptors derived from atomic or molecular properties that translate to physicochemical, topological, and surface properties of compounds. More particularly, chemical compounds can be mapped to their corresponding chemical fingerprint or molecular descriptor-based features using various methodologies that can be implemented in various environments using various software packages and/or tools that are available from electronic sources. The methodologies are not particularly limited. One example is PubChem 2D chemical structure fingerprints, which generate a binary substructure fingerprint for chemical structures. A substructure is defined as a fragment of a chemical structure. The fingerprints comprise an ordered list of binary (I/O) bits, wherein each bit represents a Boolean determination of the absence or presence of, for example, a particular element, a type of ring system, atom pairing, atom environment, etc. in a chemical structure. Other examples of methodologies include, but are not limited to, SMILES, Open Babel FP4, Molecular ACCess System (MACCS) keys, and RDKit. Examples of suitable environments for implementing the mapping include, but are not limited to, Python, Matlab, Scala, C++, Java, and Octav, among others. In some embodiments, the mapping or representing of chemical compounds to their corresponding chemical fingerprint and/or molecular descriptor can proceed in a hierarchical manner. For example, a PubChem chemical fingerprint can be generated for a chemical compound having a PubChem ID using the pubchempy package. For chemical compounds without a PubChem ID, a SMILES representation of its 2D structure can be obtained and used in the PubChem fingerprint Application Program Interface (API) to generate the chemical compound's chemical fingerprint. The Open Babel FP4 representation can be generated using the pybel package. The MACCS keys and RDKit molecular descriptors can be generated using the corresponding APIs in RDKit. See also, for example Moriwaki et al. (J. Cheminformatics, 10:4 (2018).

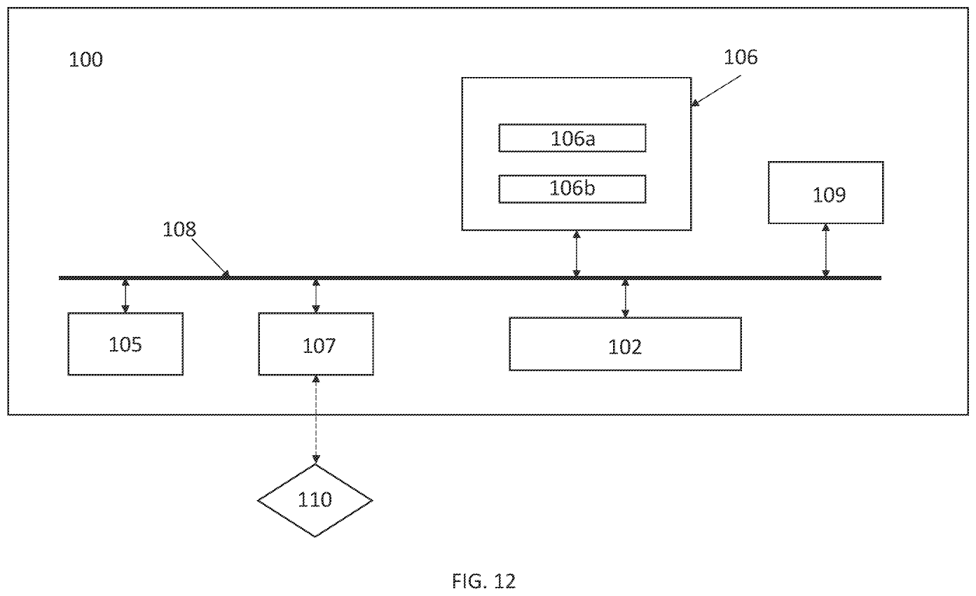

FIG. 12 is a diagram illustrating an exemplary system 100 that can be used to predict a spectrum of a target molecule. System 100 can be any suitable entity (e.g., a mobile device or a server) configurable for receiving and/or generating a descriptor (e.g., a SMILES sequence or ECFP) of the target molecule and for analyzing the molecule descriptor using a trained machine learning model to predict a spectrum (e.g., a UV-Vis spectrum) of the target molecule. System 100 can thus comprise processor(s) 102. Processor(s) 102 can represent any suitable entity or entities (e.g., hardware-based processor) for processing information and executing instructions or operations. Processor(s) 102 can be any type of processor, such as a central processor unit (CPU), a microprocessor, a multi-core processor, and the like. System 100 can further include memory 106 for storing information and instructions to be executed by processor(s) 102.

In some embodiments, memory 106 can comprise one or more of random access memory (RAM), read only memory (ROM), static storage such as a magnetic or optical disk, or any other type of machine or non-transitory computer readable medium. In some embodiments, memory 106 can store first module 106a and second module 106b instructions described hereinabove. In some embodiments, memory 106 can store data from a training molecule data set.

As further illustrated in FIG. 12, system 100 can further include user interface 105, e.g., for displaying and/or transferring the predicted spectrum and/or for importing an experimentally observed spectrum (e.g., of the target molecule and/or of one or more training molecules). Thus, user interface 105 can include a monitor or display screen to electronically display a predicted spectrum. In some embodiments, user interface 105 can include a keyboard or other suitable device for accepting user input and/or a printer. In some embodiments, such as shown in FIG. 12, system 100 can include a network or other communications interface (NIC) 107 (configured to provide communications access to entities external to system 100, e.g., other computing platforms, the internet, an internal network computer, etc.), one or more communication busses 108 for interconnecting the components of system 100, and a power supply 109 for powering the components of system 100.

In some embodiments, system 100 can include one or more additional non-volatile, non-transitory, computer readable memory devices or media (not shown) such as magnetic disk storage or persistent devices (e.g., memory means or storage means), which can optionally accessed by one or more controllers (not shown). Data in memory 106 can be seamlessly shared with the additional non-volatile memory using known computing techniques such as caching. Memory 106 or the additional non-volatile memory can include mass storage that is remotely located with respect to processor(s) 102. Thus, in some embodiments, some data stored in memory 106 or optional additional non-volatile memory can be hosted on computers that are external to system 100 but that can be electronically accessed by system 100 over an internet, intranet, or other form of network or electronic cable using network interface 107. For example, network interface 107 can be configured to send and/or receive information from internet/network 110, which can be in communication with one or more chemical database and/or client computer, e.g., to retrieve descriptors and/or spectra. In some embodiments, system 100 is a personal computer. The presently disclosed subject matter can also be performed using commercially available or custom hardware with dozens or more processors connected in parallel, at even greater speed.

In some embodiments, the additional non-volatile memory can store one or more databases that store descriptors or spectral data of one or more compounds (e.g., of a training set of molecules). In some embodiments, memory 106 stores an operating system that is configured to handle various basic system services and to perform hardware dependent tasks, and a network communications module that is configured to connect system 100 to various other computers such as remote curated data sources and/or to clients via one or more communication networks, such as the internet, other wide area networks, local area networks (e.g., a local wired or wireless network can connect the system 100 to the remote client), metropolitan area networks, and so on.

As noted above, memory 106 also can store a first module 106a and a second module 106b configured to cause processor(s) 102 to execute the various steps of a method as described herein. For example, first module 106a can be configured to cause processor(s) 102 to obtain a descriptor for one or more compounds (i.e., from an external data source, from the additional non-volatile memory, or from a remote client. In some embodiments, the descriptors are in the form of SMILES sequences, although other suitable formats can be used, such as, but not limited to, 1024- or 2048-bit ECFPs, compressed fingerprints, tokenized SMILES, or another computer readable form of a structural or physicochemical property of a molecule or a substructure thereof. First module 106a also can be configured to cause processor(s) 102 to store descriptors within a database in the additional non-volatile memory. First module 106a also can be configured to cause processor(s) 102 to assign a descriptor to a training set, based on a statistical analysis of the descriptor(s). In some embodiments, processor(s) 102 are configured to output the predicted spectrum to user interface 105 and/or network interface 107. In some embodiments, system 100 is configured to receive an experimentally observed spectrum of the target molecule, one or more training molecules, or of a mixture comprising or suspected of comprising the target molecule, e.g., directly from a UV-Vis spectrometer configured in communication with user interface 105 or via a networked source through network interface 107. In some embodiments, processor(s) 102 are configured to compare the experimentally observed spectrum of the target molecule with the predicted spectrum, e.g., using DTW, and/or to measure RMSE of the predicted spectrum. In some embodiments, processor(s) 102 are configured to output a comparison between the predicted spectrum of the target molecule and an experimentally observed spectrum of the target molecule.

In some embodiments, the first module comprises instructions that when executed by the one or more processors cause the one or more processors to retrieve or generate an extended connectivity fingerprint (e.g., ECFP6) of a target molecule. In some embodiments, the first module further comprises instructions that cause the one or more processors to compress an initially retrieved or generated ECFP by dividing the bits of the ECFP data into a plurality of groups and to convert each of the groups into a decimal value for input into the learning model. In some embodiments, the ECFP is divided into 8 groups.

In some embodiments, the first module comprises instructions that when executed by the one or more processors cause the one or more processors to retrieve or generate a SMILES sequence or string of a target molecule. In some embodiments, a tokenized SMILES sequence is retrieved or generated. In some embodiments, the second module comprises instructions that cause the one or more processors to generate a vector of weights for each character of the tokenized SMILES sequence at one or more wavelength values.

In some embodiments, the trained machine learning model is a trained model for time series data prediction and/or a trained long-short term memory (LSTM) model or machine learning model similar thereto. Other suitable learning models for use according to the presently disclosed subject matter include, but are not limited to, Random Forest, k-Nearest Neighbors, Support Vector Classification, Naïve Bayesian, AdaBoosted Decision Trees, Deep Learning, XGboost, and the like.

In some embodiments, the predicted spectrum is a UV-Vis spectrum. The predicted UV-Vis spectrum can be a spectrum that includes a predicted absorbance (e.g., a predicted relative absorbance) at a plurality of wavelengths (e.g., 2, 3, 4, 5, 6, 7, 8, 9, 10, or more wavelengths). In some embodiments, the predicted UV-Vis spectrum comprises a predicted absorbance at one or more absorption maxima and one or more absorption minima. In some embodiments, the predicted UV-Vis spectrum comprises information regarding one or more shoulders. In some embodiments, the predicted UV-Vis spectrum comprises predicted absorption values (e.g., predicted relative absorption values) for a continuous wavelength range over least 50 nm, at least 75 nm, at least 100 nm, at least 125 nm, at least 150 nm, at least 175 nm, at least 200 nm, at least 225 nm, at least 250 nm, at least 275 nm, at least 300 nm, at least 325 nm, at least 350 nm, at least 375 nm, at least 400 nm, or more. In some embodiments, the predicted spectrum is a UV spectrum. In some embodiments, the predicted spectrum is a visible spectrum. In some embodiments, the predicted spectrum comprises predicted absorption values over a wavelength range between about 190 nm to about 780 nm, about 190 nm to about 750 nm, about 190 nm to about 400 nm, or about 200 nm to about 400 nm. In some embodiments, the spectrum is a spectrum of absorption data for electromagnetic radiation other than UV and/or visible light. In some embodiments, the predicted spectrum is an IR spectrum. In some embodiments, the predicted spectrum is a NMR or MS spectrum. In some embodiments, the system is configured to output data related to the phototoxicity or color of the target molecule, e.g., based on the predicted spectrum.

As noted above, in some embodiments, the system can be further configured to receive data related to an experimentally observed spectrum of the target molecule. In some embodiments, the system can be configured to receive data related to a sample comprising a mixture of molecules comprising the target molecule or suspected of comprising the target molecule In some embodiments, the system is further configured to receive data related to an observed spectrum of the target molecule and the second module further comprises instructions for comparing the predicted spectrum with the observed spectrum, e.g., using R², RMSE, MAE. In some embodiments, the comparison is performed using Dynamic Time Warping (DTW). In some embodiments, the second module further comprises instructions for measuring the root-mean-squared deviation (RMSD) of the predicted spectrum.

In some embodiments, the system is configured to access a trained machine learning model or comprises a memory comprising a trained machine learning model. In some embodiments, the system is configured to generate the trained machine learning model. For example, in some embodiments, the system is configured to acquire (e.g., from an external data base or from a networked spectrophotometer) data for training a machine learning model, wherein the data comprises a plurality of observed spectra, i.e., wherein each of the plurality of observed spectra is the observed spectra for a different training molecule in a training set comprising a plurality of different training molecules; and wherein the data further comprises a plurality of descriptors, i.e., wherein each of the plurality of descriptors is a descriptor for a different training molecule in the training set. In some embodiments, the spectrum of one or more or each of the training molecules is correlated to the corresponding descriptor of the same molecule prior to training the model. In some embodiments, depending on the application, e.g., when the target molecule can have any one of a variety of chemical functional groups or combinations thereof, a more diverse training set can be used. In some embodiments, the training set can be optimized around a particular class of compounds, e.g., to optimize the trained model for use in predicting the spectrum of target molecules in that class.

In some embodiments, the presently disclosed subject matter provides a method for predicting a spectrum of a target molecule. In some embodiments, the predicted spectrum is a UV, Vis or UV-Vis spectrum (e.g., including predicted absorption values over a continuous wavelength range of at least about 50 nm within the UV and/or visible light wavelength range(s)). For example, in some embodiments, the UV-Vis spectrum comprises predicted absorption values over a continuous range between about 200 nm and about 400 nm.

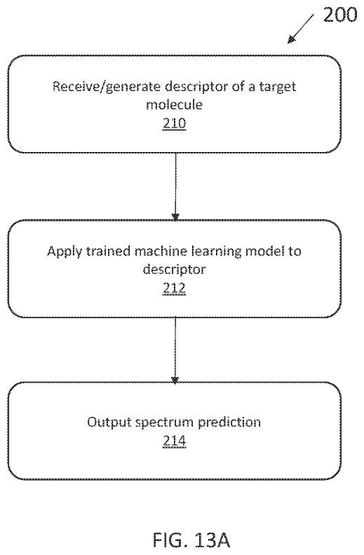

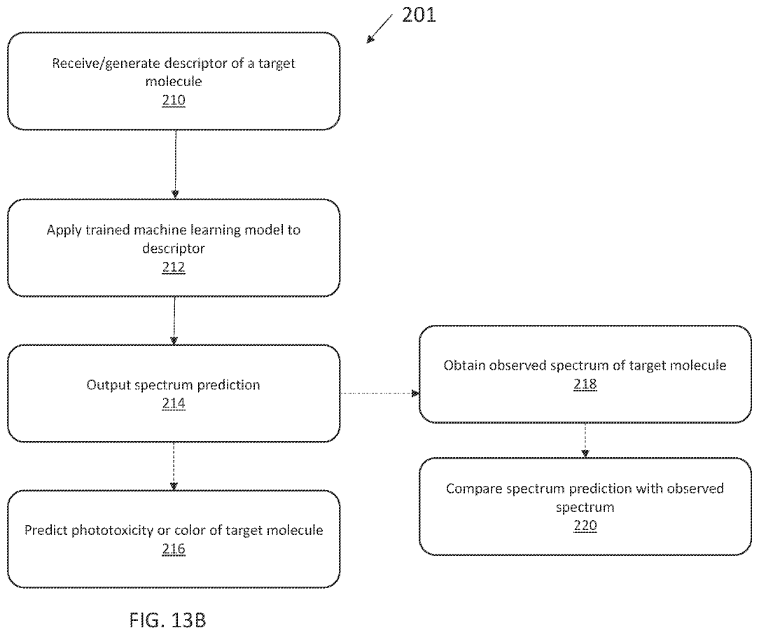

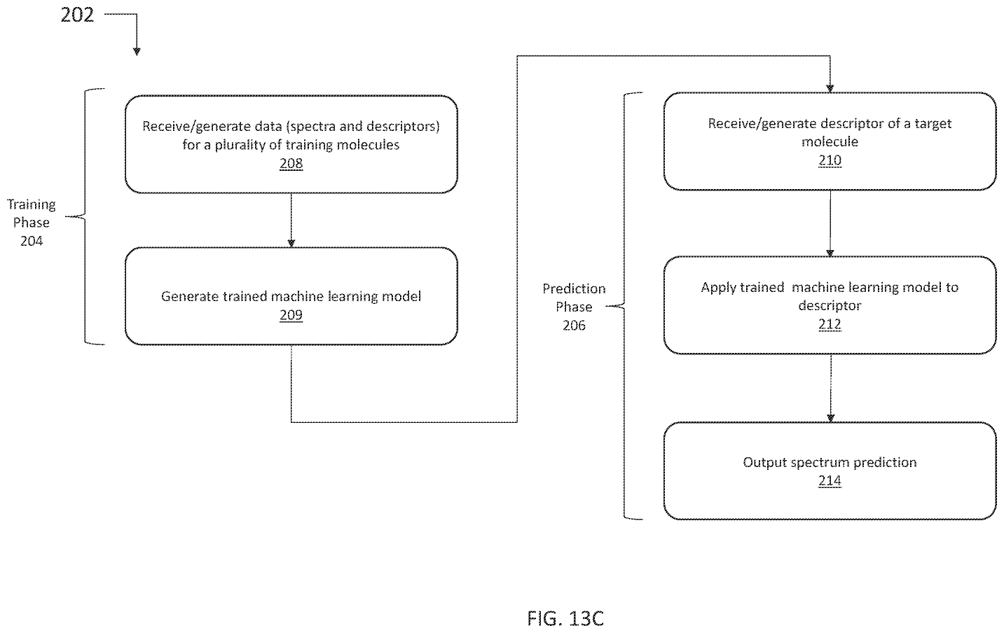

In some embodiments, the presently disclosed method can comprise method 201 of FIG. 13A, which comprises step 210 of receiving (e.g., from an external database) and/or generating (e.g., by analysis of the chemical structure of the target molecule and/or by compression or tokenization of a preliminary descriptor) a descriptor of the target molecule; and step 212 of applying a trained machine learning model to the descriptor of the target molecule with at least one processor to provide a predicted spectrum of the target molecule (e.g., wherein said trained model correlates a plurality of different descriptors to observed spectra). Continuing with FIG. 13A, method 201 can further comprise step 214 of outputting a predicted spectrum (e.g., to a display screen).

In some embodiments, the receiving and/or generating a descriptor of the target molecule of step 210 comprises receiving and/or generating a SMILES sequence, a tokenized SMILES sequence, an ECFP, or a compressed ECFP of the target molecule. In some embodiments, the receiving and/or generating comprises generating an ECFP (e.g., from a SMILES sequence). In some embodiments, the ECFP can be a 1024- or 2048-bit ECFP. In some embodiments, the ECFP can be compressed. In some embodiments, compressing comprises divided the data from the ECFP into groups and converting each group into a decimal value that can be input into the machine learning model. In some embodiments, the ECFP data can be divided into 8 groups. In some embodiments, the receiving and/or generating comprises generating a tokenized SMILES sequence. In some embodiments, e.g., as part of step 212 of FIG. 13A, the method comprises generating a vector of weights for each character in the tokenized SMILES sequence. In some embodiments, a vector of weight for each character in the tokenized SMILES sequence is generated at more than one wavelength.

The trained machine learning model can be any suitable model, such as a machine learning model for time series data prediction. In some embodiments, the model is a LSTM model or machine learning model similar thereto.

In some embodiments, as shown in method 201 of FIG. 13B, the method can optionally further comprise comparing the predicted spectrum with an experimentally observed spectrum, e.g., to determine the accuracy of the prediction. Thus, as shown in FIG. 13B, the method can optionally include step 218 of obtaining the observed spectrum of the target molecule and step 220 of comparing the observed spectrum to the predicted spectrum output in step 214. In some embodiments, the comparison of step 220 can be done visually. In some embodiments, step 220 can involve a suitable statistical method for comparison (i.e., to more quantitatively determine the accuracy of the predicted spectrum). In some embodiments, the comparison can be performed using DTW. In some embodiments, the method comprises measuring the RMSD of the predicted spectrum.

In some embodiments, also as shown in FIG. 13B, method 201 can optionally include an additional prediction step 216 for determining another property of the target molecule. For example, when the target molecule is a possible drug candidate, step 216 can comprise predicting the phototoxicity of the target molecule. In this regard, molecules that absorb light in a range between 290 nm and 700 nm with a molecular extinction coefficient (MEC) greater than 1000 Lmol⁻¹ cm⁻¹ are understood in the field to be potentially photoreactive (see ICH S10, “Photosafety Evaluation of Chemicals” (2012)), e.g., by being able to generate reactive oxygen species when excited by light in that range Thus, if the λₘₐₓ from the predicted spectrum is below this threshold, the molecule can be predicted to be unlikely to have phototoxicity. In some embodiments, such as when the target molecule is a possible dye or colorant molecule, step 216 can comprise providing a visible spectrum that provides information regarding the color of the target molecule.

In some embodiments, as shown in FIG. 13C, the presently disclosed method can be a method such as method 202, comprising training phase 204 in addition to prediction phase 206, which as shown in FIG. 13C includes the same steps 210, 212, and 214 as described hereinabove for method 200 of FIG. 13A. Training phase 204 of method 202 can be performed prior to prediction phase 206 (or at least prior to step 212 of prediction phase 206) to generate a trained machine learning model for use in predicting the spectrum of the target molecule. Training phase 204 can include step 208 for receiving and/or generating data such as spectra (e.g., UV-Vis spectra) and descriptors for a plurality of different training molecules. For example, step 208 can comprise: acquiring or generating a plurality of observed spectra, wherein each of the plurality of observed spectra is the observed spectra for a different training molecule in a training set comprising a plurality of training molecules; and acquiring or generating a plurality of descriptors, wherein each of the plurality of descriptors is a descriptor for a different training molecule in the training set. In some embodiments, the spectra and descriptors can be retrieved from a memory or from an external database. In some embodiments, the spectra are generated experimentally and input into a system of the presently disclosed subject matter. In some embodiments, step 208 comprises compressing or tokenizing the descriptors. Continuing with FIG. 13C, training phase 204 further comprises step 209 of generating the trained machine learning model using the data received or generated in step 208. In some embodiments, step 208 comprises correlating one or more of the spectra (or each of the spectra) to the corresponding descriptor (i.e., the descriptor of the same molecule) prior to using the data to train the machine learning model in step 209.

In some embodiments, the presently disclosed subject matter provides a method for detecting a target molecule in a mixture of molecules. For example, the mixture of molecules can be an aliquot of a reaction mixture of an organic chemical functional group transformation reaction, e.g., as part of the synthesis of a possible drug candidate and/or natural product; a sample from a combinatorial library that comprises a mixture of molecules; a biological sample (e.g., a cell extract or a plasma or blood sample), an industrial wastewater stream; or an environmental sample (e.g., a water sample or a soil extract sample). The target molecule can be a drug candidate, a synthetic intermediate, a metabolite, a dye or other colorant, a pesticide, or an environmental toxin. In some embodiments, the method comprises (a) obtaining a spectrum of the mixture of molecules (e.g., obtaining a UV-Vis spectrum of the mixture of molecules), and (b) comparing the spectrum from (a) with a predicted spectrum of the target molecule obtained by performing a method of the presently disclosed subject matter and/or using a system of the presently disclosed subject matter. In some embodiments, the spectrum obtained in step (a) is a spectrum obtained from an aliquot of a chromatography eluant. In some embodiments, the spectrum obtained is from an aliquot of a synthetic reaction mixture or of a chromatography eluant of a synthetic reaction mixture. In some embodiment, the chromatography eluant is a HPLC eluant.

In some embodiments, the spectrum obtained in step (a) is a spectrum obtained from a sample present in a well of a microarray or microwell plate. In some embodiments, the microarray or microwell plate comprises a plurality of wells and each of the plurality of wells contains a sample that is different from the sample present in any other of the plurality of wells (i.e., contains a different molecule or different combination of molecules than the sample present in any other of the plurality of wells). Accordingly, in some embodiments, the presently disclosed subject matter provides a method for detecting a target molecule in a mixture of molecules, wherein the method comprises: (a) providing a microarray or microwell plate comprising a plurality of wells, wherein each of the plurality of wells contains a sample that comprises one or more molecules, and wherein each of the plurality of wells contains a sample that comprises a different molecule or combination of molecules than in a sample present in any other of the plurality of wells; (b) obtaining a spectrum (e.g., a UV-Vis spectrum), of the sample present in each of a plurality of wells of the microarray or microwell plate, thereby obtaining a plurality of spectra; and (c) comparing the spectra from (b) with a predicted spectrum of the target molecule, optionally a predicted UV-Vis spectrum of the target molecule, wherein said predicted spectrum is obtained by performing a method of the presently disclosed subject matter and/or using a system of the presently disclosed subject matter.

In some embodiments, the presently disclosed subject matter provides a non-transitory computer readable medium comprising computer executable instructions embodied in a computer readable medium that when executed by a processor of a computer control the computer to perform steps comprising: receiving and/or generating a descriptor of the target molecule (e.g., a SMILES sequence, a tokenized SMILES sequence, an ECFP or a compressed ECFP of the target molecule); and applying a trained machine learning model to the descriptor to predict a spectrum of the target molecule.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Materials and Methods

Libraries of molecules from an internal collection of SRI International (Menlo Park, California, United States of America) were run after a dilution to 200 μM with methanol, and 3 μL injections from conical well plates sealed with either film or plate mats. Experiments with OTAVA library plates (OTAVA Chemicals, Concord, Ontario, Canada) used the plate-sealing, inert plastic mats, and DMSO for dilutions.

### UV-Vis Spectra Generation

The equipment included a Thermo LCQ Fleet ion trap mass spectrometer (MS) (Thermo Finnigan LLC, San Jose, California, United States of America) paired with a ultra performance liquid chromatography (UPLC) system sold under the tradename DIONEX™ ULTIMATE™ U3000 (ThermoFisher Scientific, Waltham, Massachusetts, United States of America) featuring a HPG-3200RS binary pump, a DAD-3000RS diode array detector, and a WPS-3000TBRS autosampler (all from ThermoFisher Scientific, Waltham, Massachusetts, United States of America). The features of each are described below:

WPS-3000 TBRS Autosampler: Provides high precision and accuracy even at ultra-high-performance liquid chromatography (UHPLC) pressures up to 1034 bar, with corrosion-resistant sample flow path for highest integrity of biosamples; biocompatible analytical scale split-loop sampler with sample thermostatting (4-45° C.), injection volume 0.2-25 μL (field-upgradable to 500 μL), 3 segments for vial racks/wellplates; contams WPS-3000TBRS module and installation accessories (ship kit; includes 3×48 pos. sample racks).

HPG-3200RS binary pump: High-Pressure Mixing Gradient Pump. Supports high-speed and high-resolution applications as well as standard HPLC workflows. Biocompatible, settable flows of 0.001-8 mL/min and pressures up to 1034 bar (with a flow rate of >5 mL/min, pressure range decreases linearly down to 800 bar), 400 μL mixer, 400 μL GDV, 2 solvent channels; contains UltiMate 3000 HPG-3200RS module and installation accessories (ship kit) including RS system tubing.

DAD-3000RS diode array detector: Offers high-resolution and a 1024-element diode array with up to 200 Hz data rate, with a wavelength range of 190 to 800 nm. Provides variable slit width. The detector was equipped with a FLOW CELL, SEMI-MICRO, 2.5UL, PEEK, DAD/MWD Semi-Micro Flow Cell for DAD-3000 and MWD-3000 Series, PEEK 2.5 μL Volume, 7 mm Path Length.

### Machine Learning Methods

The spectra prediction makes use of a Deep Learning Machine Learning algorithm called LSTM (Long-Short Term Memory) model¹⁰. This model is mainly used for time series data prediction. The application to UV-Vis spectra prediction can be considered as a form of “time series” with lambda as the x-axis as it is proportional to time linearly (using Wavelength(lambda(λ))=c(speed of light)*t(time)). A wavelength window from 220 to 400 nm was used. The extended connectivity (ECFP6, also known as Morgan Fingerprint with diameter 3) fingerprint for each molecule calculated from the SMILES using RDKit library in python (available online at rdkit.org) cheminformatics library was also used. This fingerprint array is composed of binary bits 1 and 0 and the array's total size set is 2048 bits (default value). These 2048 bits were divided into groups of 256 bits resulting in a total of 8 groups for a molecule (8-bit). These groups are then converted to base 10 integers (i.e., to decimal values). The LSTM model takes these 8 decimal values as parameters along with the wavelength value (generated in 1 nm increments for a spectra) for each molecule to build a model. The test column is the absorbance value to be predicted. The model is usually trained using a 70:15:15 (train: test: validation) split. These splits are identified on the basis of clustering based on molecular structural similarity using minimum description length (MDL) descriptors (using Discovery Studio, Biovia, San Diego, California, United States of America). The LSTM model has:-4 layers with return sequence set to True and activation function=‘relu’; 3 Dense layers with a final layer being the output layer; The model makes use of Adam Optimizer and loss is measured using the Mean Square Error while training. The number of epochs the code runs for is 2500 with batch size=5.

### Server Details

Computational Servers included the following components: Supermicro EATX DDR4 LGA 2011, Intel Computer CPU 2.1 8 BX80660E52620V4, Crucial 64 GB Kit (16GBx4) DDR4 2133 (PC42133) DR×4 288 Pin Server Memory CT4K16G4RFD4213/CT4C16G4RFD4213, 2× EVGA Geforce GTX 1080 Ti FOUNDERS EDITION GAMING, 11 GB GDDR5X, 2.5 Inch Solid State Drive Intel 730 SERIES SSDSC2BP480G410, WD Gold 4 TB Datacenter Hard Disk Drive 7200 RPM Class SATA 6 Gb/s 128 MB Cache 3.5 Inch WD4002FYYZ and Supermicro 920 Watt 4U Server. The following software modules were installed: nltk 3.2.2, scikit-learn 0.18.1, Python 3.5.2, Anaconda 4.2.0 (64-bit), Keras 1.2.1, Tensorflow 0.12.1, Jupyter Notebook 4.3.1.

### Spectra Comparison Measures and Metrics

For each molecule, the RMSD and the DTW were calculated using the scikit library in python. DTW is a distance measure technique which allows a non-linear mapping between two signals by minimizing the distance between the two¹¹. DTW works by constructing an n-by-m matrix where the i^{th}, j^{th} element of the matrix corresponds to the squared distance, d(qᵢ, q_{c})=(qᵢ, q_{c})² of two time series, Q=q₁, q₂, . . . , qₙ and C=c₁, c₂, c₃, . . . , cₘ. DTW finds the minimum cost path through the matrix with constraints, in essence following a path that warps through time. This method is flexible, allowing two time series that are similar but locally out of phase to align non-linearly. DTW is a well-known solution for time series alignment in multiple domains, and is often the best solution or within a small margin of the best solution for any problem in time series classification¹².

### Results

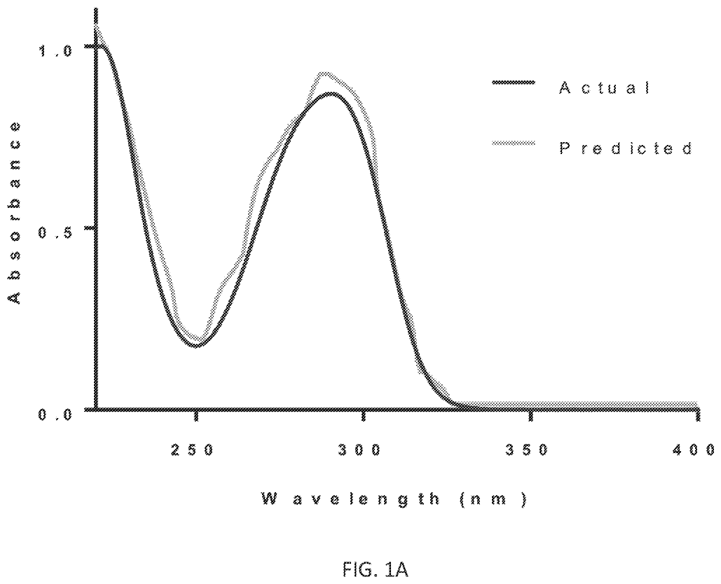

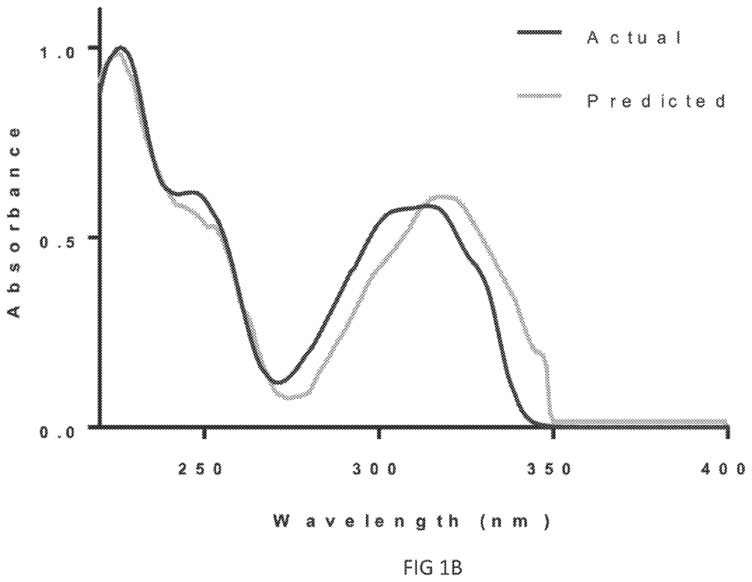

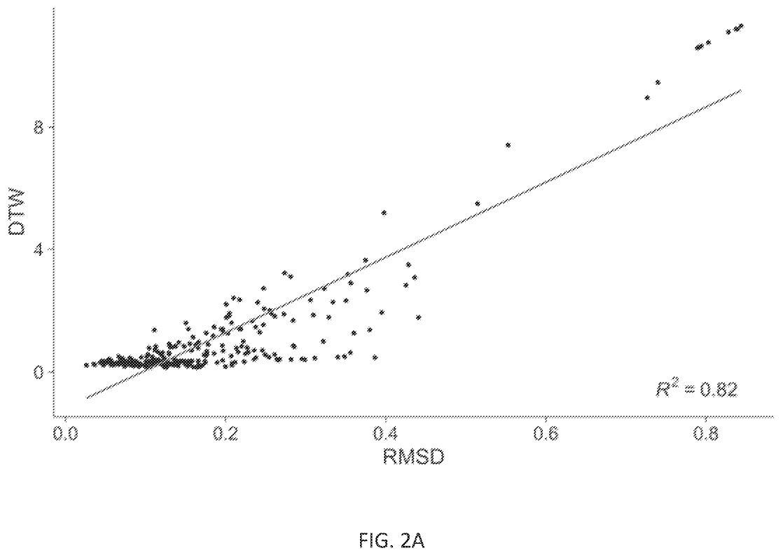

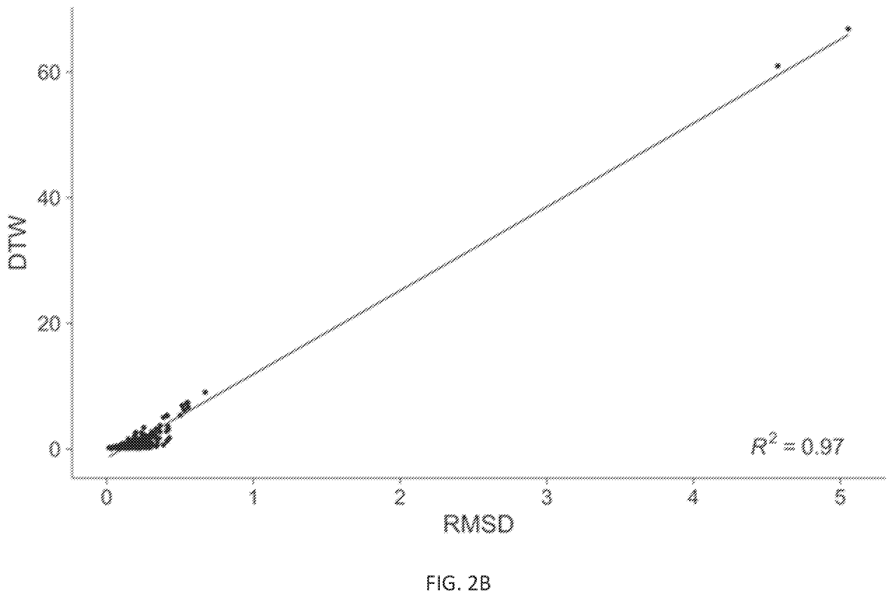

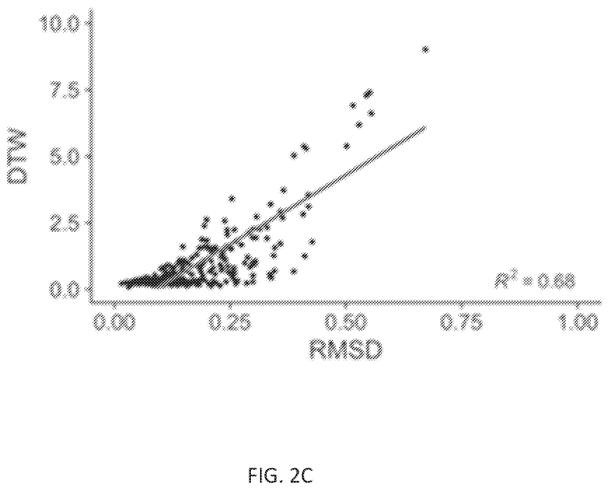

An array of computational experiments were performed to evaluate the effect of descriptor bit length as well as training and test set evaluations. See Table 1, below. For example, on a training set of more than 154 molecules and a test set of 58 molecules, an average RMSD of ˜0.24 was demonstrated. On a larger training set of 677 and a test set of 291 molecules, the RMSD for the test set was 0.18. Examples of individual spectra are shown in FIGS. 1A and 1B. DTW, a second spectra comparison measure which is much more robust to matching spectra shapes even if they are out of phase¹³, provided an interpretable method to compare predicted and observed spectra to access machine learning prediction quality and correlates with RMSD (R²>0.8). See FIGS. 2A-2C. The RMSD values were comparable or better than that achieved by DFT (when comparing the smallest model, as average RMSD was ˜0.32) with the advantage that once the machine learning model is trained, the predicted spectra for a new molecule can be predicted in seconds.

TABLE 1
Summary of training and test set information for UV-Vis spectra prediction using LSTM.

| Number of | Number of | | | | | | |
|---|---|---|---|---|---|---|---|
| Training | Test | Average | Minimum | Maximum | Average | Minimum | Maximum |
| Compounds | Compounds | RMSD | RMSD | RMSD | DTW | DTW | DTW |
| 47 | 1 | 0.34 | 0.036 | 0.76 | | | |
| 154 | 58 | 0.24 | 0.04 | 0.55 | | | |
| 677 | 291 | 0.18 | 0.026 | 0.84 | 1.08 | 0.15 | 11.32 |
| 690 | 299 | 0.21 | 0.027 | 0.67 | 1.36 | 0.09 | 66.87 |

Using a machine learning approach for UV-Vis spectra prediction would be expected to require a very large training dataset of diverse spectra to cover chemistry space. However, according to the presently disclosed subject matter it has been demonstrated that with relatively small numbers of molecules, good levels of accuracy of prediction can be obtained (based on the average RMSD˜0.2 when trained and tested on hundreds of molecules). The presently disclosed method provides the ability to score libraries of millions-billions of molecules and, thus, more rapidly and reliably identify molecules with a desirable UV-Vis spectrum. The presently disclosed subject matter can have applications for new dye discovery (e.g. for prediction of colors), organic chemistry reaction monitoring, and phototoxicity prediction, among other applications²⁻⁹. In addition, alternative measures, such as DTW, can also help in assessment of observed and predicted spectra and it is expected that these scores can be used to assist in training the machine learning model cost functions.

### Example 2

### Compound Libraries

Absorbance spectra were acquired for a diverse set of 393 compounds (from an internal collection of SRI International, Menlo Park, California, United States of America) and from a collection of 596 compounds purchased from OTAVA Chemicals (MMP2 Targeted Library, OTAVA Chemicals, Concord, Ontario, Canada). These two collections formed “Dataset I”. Compounds were diluted to 200 mM with methanol or DMSO and arrayed in 96-well plates for analysis by HPLC with spectrum acquisition. The MicroSource Spectrum screening compound library of 2222 compounds (MicroSource Discovery Systems, Inc., Gaylordsville, Connecticut, United States of America) was a generous gift from Dr. Ethan Perlstein, (Perlara PBC, San Francisco, California, United States of America). This library formed “Dataset II”.

### UV-Vis Spectrum Acquisition.

Compounds for Dataset I were analyzed by HPLC using a UPLC system sold under the tradename DIONEX ULTIMATE U3000 (ThermoFisher Scientific, Waltham, Massachusetts, United States of America) equipped with a LCQ Fleet ion trap MS (Thermo Finnigan LLC, San Jose, Califomia, United States of America), a DAD-3000RS diode array detector (DAD; ThermoFisher Scientific, Waltham, Massachusetts, United States of America), and a Cis column. The mobile phase was water-acetonitrile-0.1% formic acid, with an acetonitrile gradient.

The retention time for the compound of interest in each chromatographic run was determined from the extracted ion chromatogram (XIC). The XIC was scanned for the largest peak at the expected mass. When found, the peak was fit with a Gaussian and was accepted if it met constraints for lineshape (Gaussian full width at half maximum (FWHM)<0.1) and elution time greater than the void volume of 1.2 min. This process eliminated compounds that had no mass response or potential co-elution with sample impurities. It resulted in inclusion of spectra for 949 compounds from the starting set of 989. For each accepted chromatogram, an empirically determined time-offset was applied to extract the UV-Vis spectrum (200 nm to 800 nm) for that compound from the DAD data.

Background due to HPLC mobile phase absorption was subtracted from each spectrum. Due to the gradient in acetonitrile concentration, the background spectrum depended on the elution time of the analyzed compound. To assess the background at the relevant elution time for each compound, the minimum signal at each wavelength was extracted from the set of all spectra collected at that elution time for a given plate of compounds. The minimum signal from the set was taken to be the background without contribution from analytes or compound-specific impurities. The resulting inferred background spectrum for the relevant elution time was subtracted from the measured spectrum of each compound. The background-subtracted spectra were truncated (220 nm to 400 nm) and scaled by setting the minimum absorbance to zero and normalizing to a maximum absorbance of 1.0.

Compounds for Dataset II were obtained as 10 mM solutions in 100% DMSO. Each compound was diluted 50-fold (to 200 mM and 2% DMSO) with water and transferred to black, clear-bottom microplates sold under the tradename UV-STAR® (Greiner Bio-One GmbH, Frickenhausen, Germany). The UV absorption of each compound was read in a multi-mode microplate spectrophotometer sold under the tradename SPECTRAMAX® iD5 (Molecular Devices LLC, San Jose, California, United States of America) from 230 nm to 400 nm in 1 nm increments. The resulting spectra were scaled by setting the minimum absorbance to zero and normalizing to a maximum absorbance of 1.0.

### Dataset Preparation

SMILES for the Dataset I compounds were exported from a Collaborative Drug Discovery (CDD) vault informatic platform (Collaborative Drug Discovery, Burlingame, California, United States of America). Molecules were prepared as follows: Salts were removed and molecules were neutralized if possible. Molecules were converted into their canonical SMILES format using the opensource cheminformatics software RDKit. Duplicates were then removed from the dataset.

### Machine Learning Methods

Spectrum prediction makes use of a Deep Learning Machine Learning algorithm called LSTM (Long-Short Term Memory) model.¹⁰ See FIGS. 3A-3C. Wavelength windows from 220 to 400 nm for the spectra from Dataset I and 230 to 400 nm for spectra from Dataset II (due to the wavelength limitations of the spectrophotometer) were used. For input, four different data representations were considered: 1024-bit or 2048-bit ECFP6 fingerprint, a compressed fingerprint, and the tokenized SMILES string as parameters along with the full wavelength values for each molecule to build a model.

More particularly, the connectivity fingerprint 6 (ECFP6, also known as Morgan Fingerprint with diameter 3) fingerprint for each molecule was calculated from the SMILES. This fingerprint array is composed of binary bits 1 and 0. To create a compressed fingerprint, 2048-bit ECFP6 were generated and divided into groups of 256, resulting in a total of 8 groups for a molecule (compressed fingerprint). These groups are then converted to base 10 integers (i.e., to decimal values). Fingerprints were input as features as floats of the molecule directly to the first LSTM layer.

For the SMILES-based model and Seq2Seq model, each unique character in the SMILES vocabulary was represented as a separate integer, except for Br and any closed brackets notation, which were given their own separate integers. A beginning (<B>) token was added in the front of each SMILES, and an end-of-sequence token (<EOS>) was added at the end of each SMILES string, each with their own unique integer representation. Each SMILES string was thus tokenized by converting into an integer representation which was used as the input sequence into the spectra models. The test column is the absorbance value to be predicted. The model was trained using a randomized 70.15:15 (train: test: validation) split.

Two general model architectures were used. The first is composed of 4 LSTM layers (2048, 1024, 512, and 156 hidden units, respectively) using relu activation with dropout layers in between each LSTM layer. This is followed by one dense layer of 128 units and a final dense layer being the output layer corresponding to all 171 or 181 wavelength values. The SMILES-based LSTM model has an additional embedding layer (output size: 1024) to accept the integer-based tokenized SMILES representation. The model makes use of Adam Optimizer and loss is measured using the MAE while training. The code runs for 300 epochs with batch size=10. The second model architecture is based on Seq2Seq model with Luong attention.^{31,32} An embedding layer (output size: 1024) followed by a 3-layer bi-directional LSTM with 512 hidden units was used for the encoder. Without being bound to any one theory, it was believed that the encoder would benefit from the relationship between atoms and bonds from both a forward and backward direction. A Luong attention mechanism was incorporated using dot-product to compute the attention score. For the decoder, one dense layer of size 1024 units that accepted a single float value was used followed by 6 layers of 1024 hidden units of LSTM layers. After the LSTM layers, a single dense layer (1024 units) was the output layer for predicting a single wavelength value. Use of bi-directional LSTMs did not improve the model's predictive abilities in the decoder, so regular LSTM layers were used to save computational cost. The model was trained as follows. First, SMILES are tokenized (as described herein above). After tokenization, each integer-representation of the SMILES string was fed in one at a time into the encoder, generating a hidden vector for each input in the tokenized SMILES string. The hidden vectors for the forward and reverse LSTMs in the bi-directional LSTM layers were concatenated. After the entire SMILES string was input, 0.0 was used as a starting value for the decoder. This value was fed through the decoder LSTM layers and output a hidden vector. An attention score was computed using the entire encoder hidden output and the current decoder hidden output using the dot-product. This attention score was fed through a softmax layer to compute the attention weight vector for each input value. Finally, the attention weight vector and hidden state of the decoder were combined to create a context vector, which was fed through a final linear layer for the single wavelength prediction score.

To accelerate learning, teacher forcing was used to begin model training, in which the decoder is given the correct previous value as input to decode the next value regardless of the output at each decoder time step. Scheduled sampling was used to reduce the amount of teacher forcing over time, starting at 100% teacher forcing (for each input, teacher forcing is used), and reducing the chance to use teacher forcing by 10% every 2 epochs (starting at epoch 3, teacher forcing would have a 90% occurring for each training input sequence in the batch; at epoch 5, teacher forcing would be 80%; etc.) until teacher forcing was no longer used (0%, epoch 23). Instead, for each decoder timestep, the predicted wavelength value at the previous time steps were input as the new start value for the next step of the decoder. The model was trained using early stopping based on the validation loss (patience=5), a batch size of 64, with an Adam optimizer (learning rate of 1e-4 and a weight decay of 1e-4). The model finished training after 115 epochs.

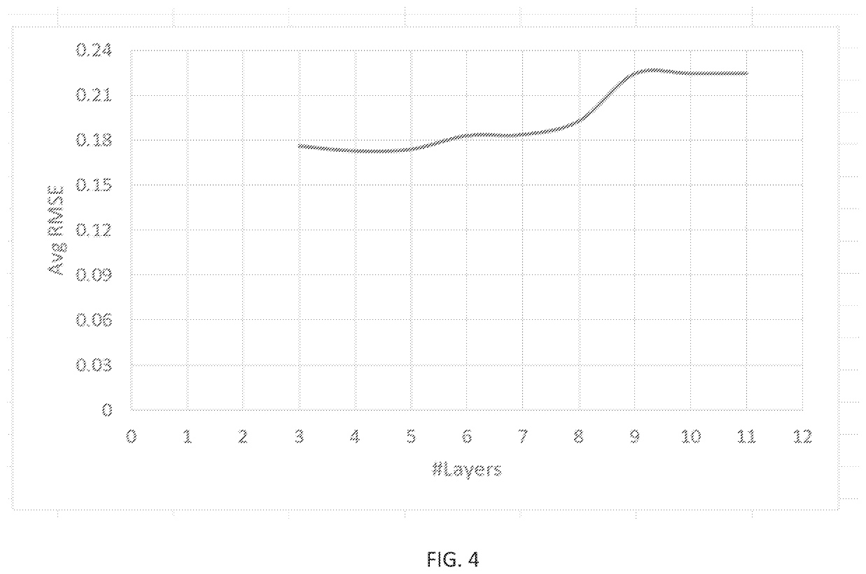

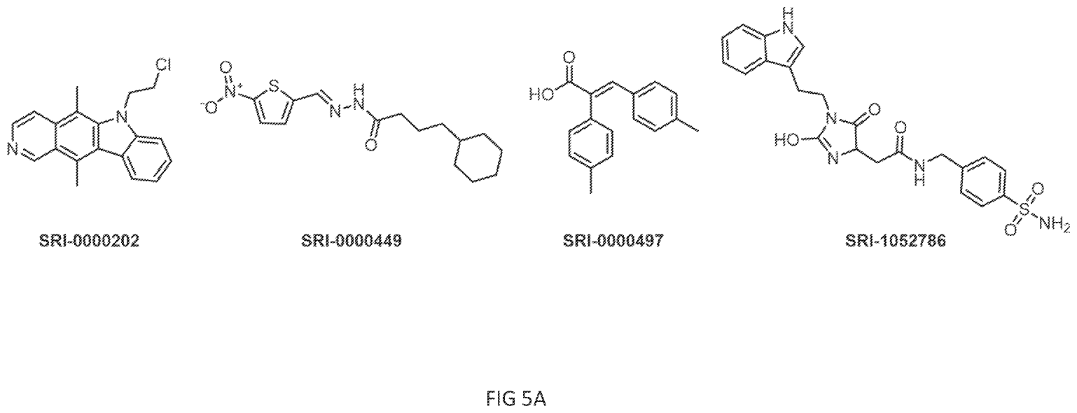

To identify the optimal parameters for the model, GridSearchCV was used in Scikit Learn on the training and in evaluating results on the evaluation set. For the model, the optimization was run 2 times. The grid search parameter, cv_folds, was set to 3 each time. The first time it was run to identify the number of hidden layers that the model should have. Code was run for layers: 3, 4, 5, 6, 7, 8, 9, 10, 11. Out of these, the model's best performance was seen when the number of layers was equal to 4. This parameter is where the model had the lowest average RMSE. See FIG. 4. The second time, it was used to identify the appropriate learning rate and dropout rate for the model. The following combinations were tried: learning rate: 0.1, 0.01, 0.001 and dropout rate: 0.1, 0.3, 0.4. Out of the above combinations, the lowest RMSE was observed for learning rate=0.01 and dropout rate=0.3 or 0.4. However, when the model was trained with these parameters, it was seen that the model was only predicting a single curve as the best fit for all the training curve. Therefore, the second-best parameter combination was chosen, which was Ir=0.001 and dropout rate=0.3. All the models reported have the following parameters: number of layers: 4, learning rate: 0.001, dropout rate: 0.3. See Table 2, below, and FIG. 4.

TABLE 2
Machine learning parameter optimization.

| Parameter_learn- | | | |
|---|---|---|---|
| ing_rate | parameter_r | std_test_score | rank_test_score |
| 0.1 | 0.1 | 0.47 | 4 |
| 0.1 | 0.3 | 0.46 | 6 |
| 0.1 | 0.4 | 0.47 | 9 |
| 0.01 | 0.1 | 0.47 | 4 |
| 0.01 | 0.3 | 0.02 | 1 |
| 0.01 | 0.4 | 0.01 | 1 |
| 0.001 | 0.1 | 0.05 | 8 |
| 0.001 | 0.3 | 0.15 | 3 |
| 0.001 | 0.4 | 0.12 | 7 |

### Server Details

Computational Servers included the following components: Supermicro EATX DDR4 LGA 2011, Intel Computer CPU 2.1 8 BX80660E52620V4, Crucial 64 GB Kit (16GBx4) DDR4 2133 (PC42133) DR×4 288 Pin Server Memory CT4K16G4RFD4213/CT4C16G4RFD4213, 2× EVGA Geforce GTX 1080 Ti FOUNDERS EDITION GAMING, 11 GB GDDR5X, Intel 730 SERIES 2.5 Inch Solid State Drive SSDSC2BP480G410, WD Gold 4 TB Datacenter Hard Disk Drive 7200 RPM Class SATA 6 Gb/s 128 MB Cache 3.5 Inch WD4002FYYZ and Supermicro 920 Watt 4U Server. The following software modules were installed: nltk 3.2.2, scikit-learn 0.18.1, Python 3.5.2, Anaconda 4.2.0 (64-bit), Keras 1.2.1, Tensorflow 0.12.1, Jupyter Notebook 4.3.1.

t-SNE Visualization

t-SNE³⁹ embeds data into a lower-dimensional space. 1024 ECFP6 fingerprints were generated for all compounds. The 1024 bit fingerprints were then embedded into a 2-dimensional vector using t-SNE. All t-SNE values were generated using the scikit-learn library in python with default hyperparameters (n_components=2, perplexity=30, early exaggeration=12.0, learning rate=200, n_iter=1000)

### Clustering of Spectra

To cluster spectra, the package tsclust in R was used.^{47,48} Spectra were clustered using shape-based distance clustering.⁴⁹ To determine the number of optimal clusters that exist in the data, the silhouette method was performed.⁵⁰ Briefly, the silhouette method determines how similar each datapoint is to its own cluster (intra-cluster distance) in comparison to how similar the datapoint is to other clusters (inter-cluster distance). From 1 to 10, k-means clustering was performed on the spectra dataset and the silhouette method was performed to measure inter-cluster distance vs. intra-cluster distance. The average silhouette is calculated for each datapoint, and the k-means clustering with the highest average silhouette value is considered the optimal number of clusters to portion the data into.

### Spectrum Comparison Measures

For each compound, the actual and predicted spectra were compared and the statistics were calculated using the scikit-learn library in python. DTW works by constructing an n-by-m matrix where the i^{th}, j^{th} element of the matrix corresponds to the squared distance, d(qᵢ, q_{c})=(qᵢ, q_{c})² of two time series, Q=q₁, q₂, . . . , qₙ and C=c₁, c₂, c₃, . . . , cₘ. DTW finds the minimum cost path through the matrix with constraints, in essence following a path that warps through time.

### Results

The presently disclosed subject matter (also referred to herein as UV-ADVISOR™) provides in some embodiments a new tool to enable a scientist to obtain predicted UV-Vis absorption spectra for input molecules using standard structure representations such as structure data file (SDF)²⁹ and SMILES.³⁰ Initially, several feed forward machine leaning models were tested, however they all failed to converge. The machine learning model built from a Long Short-Term Memory (LSTM) network architecture to predict relative absorbance at wavelengths within a trained range (see FIGS. 3A-3C) performed the best. To cover a wider range of applicability, two models were trained, each with a different dataset which covers different chemical property space. See FIG. 3B. Spectra for the compounds of Dataset I were obtained with a PDA detector interfaced with a HPLC, the elution time of each sample compound being judged by its initial detection with an in-line mass spectrometer. Dataset II was generated from a commercially obtained collection of drugs. Spectra for these compounds were obtained with a spectrophotometer using a multi-well plate format. Generating two datasets using two distinct methods provided for the demonstration of the wider applicability of UV-Vis based models, as UV-Vis spectra can often be distinct based on conditions such as the solvent composition and the pH. The spectra in both data sets were baseline corrected (minimum value in wavelength range offset to 0) and normalized (maximum value in wavelength range set to 1). For each model, 70% of the compounds were used for training, 15% for validation, and 15% for testing. A first set of models used LSTM layers to read SMILES sequences or an ECFP6 fingerprint. See FIG. 3C, left. A second model architecture was also used, taking advantage of recent advancements in using encoder-decoder architectures^{31,32} with an attention mechanism for language translation. See FIG. 3C, right. This second network architecture is motivated by approaching spectrum prediction as a sequence to sequence (Seq2Seq) translation problem between a chemical structure (represented by SMILES string) and a wavelength sequence output. The final models are readily accessible through a web interface (available at online collaborationspharma.com/uvadvisor), where the user can input a structure in 2D or SMILES format, and the presently disclosed UV-ADVISOR™ tool outputs the predicted spectrum as a graph or in .csv format.

### Provision of Accurate Spectrum Predictions

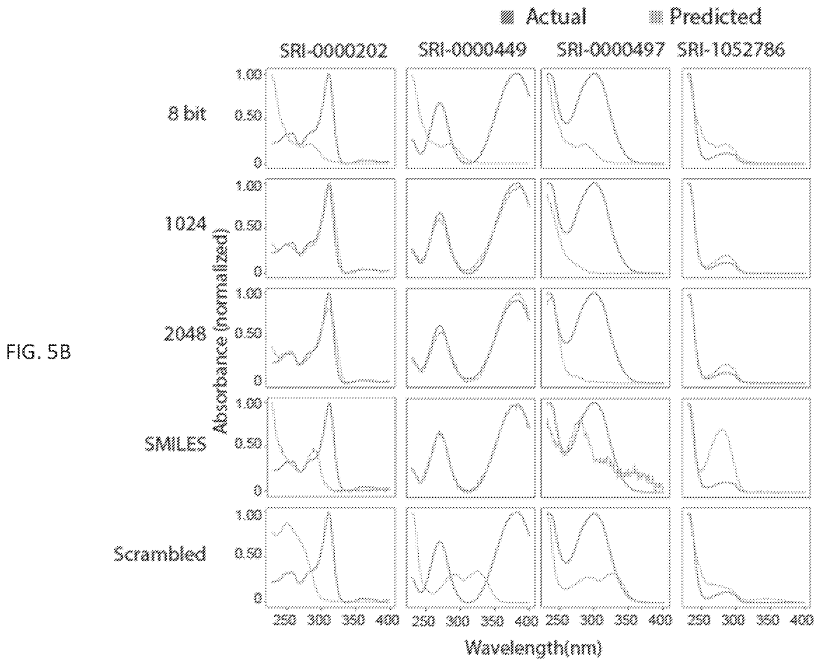

Models generated using Extended Connectivity Fingerprint Diameter 6 (ECFP6)^{33,34} molecular representations as inputs to the LSTM network produced high-quality predictions of spectra for test compounds. Representative examples from the model using Dataset I are shown in FIGS. 5A and 5B. Many of the predictions accurately render absorption maxima, minima, and shoulders and good approximations of relative absorption across the wavelength range of the spectra. The best predicted spectrum had a RMSE of predicted versus measured spectra of 0.005 (SRI-1053215). Qualitatively, RMSE values of less than 0.10 were assessed as “excellent”, values less than 0.20 as “good”, and anything at or above 0.25 as a “poor” prediction. Comparable prediction accuracy was obtained, as judged by RMSE (see Table 3, below), with a model that used 2048-bit or 1024-bit ECFP6 descriptors (see Methods). The median RMSE for both sets of predictions is ˜0.17. However, further compression of the fingerprint resulted in substantial degradation of the prediction quality (median RMSE=0.21). Using tokenized SMILES as the molecular representation produced predictions of quality comparable to those produced with the uncompressed ECFP6 (median RMSE=0.17). Using a Seq2Seq model resulted in the best predictive model (as judged by median RMSE=0.15). Training the model with scrambled data, in which the compounds are paired randomly with spectra from the dataset, resulted in poor predictions as one would expect. The average median RMSE for predictions made with LSTM models trained with three randomly scrambled sets using 2048-bit ECFP6 was degraded to 0.25. Comparison of this performance metric with the that of the trained model with the correctly paired spectra and compounds confirms that the model has successfully learned structure-spectrum relationships. Certainly, there are other performance metrics which could be considered, for example peak-wavelength predictions.

TABLE 3
Summary of Dataset I training (N = 649) and test
set (N = 150) information for UV-Vis spectrum
prediction using LSTM or Seq2Seq with attention (Scrambled
average of N = 3, ±standard deviation).

| | Median | Median | Median | Median RMSE | Median |
|---|---|---|---|---|---|
| Model | RMSE | DTW | R² | derivative | MAE |
| 8-bit | 0.206 ± | 0.705 ± | 0.420 ± | 0.016 ± | 0.123 ± |
| Compressed | 0.141 | 1.468 | 1.446 | 0.009 | 0.115 |
| ECFP6 | | | | | |
| 1024-bit | 0.169 ± | 1.029 ± | 0.626 ± | 0.013 ± | 0.119 ± |
| ECFP6 | 0.132 | 1.232 | 1.166 | 0.010 | 0.106 |
| 2048-bit | 0.169 ± | 0.760 ± | 0.546 ± | 0.015 ± | 0.121 ± |
| ECFP6 | 0.143 | 1.395 | 1.266 | 0.010 | 0.112 |
| SMILES | 0.166 ± | 0.710 ± | 0.629 ± | 0.025 ± | 0.106 ± |
| | 0.140 | 1.187 | 1.207 | 0.015 | 0.118 |
| Seq2Seq | 0.154 ± | 0.558 ± | 0.680 ± | 0.018 ± | 0.091 ± |
| | 0.144 | 1.268 | 1.230 | 0.020 | 0.12 |
| Scrambled | 0.250 ± | 1.162 ± | 0.124 ± | 0.019 ± | 0.170 ± |
| Average | 0.134 | 1.429 | 1.434 | 0.009 | 0.113 |

### Training on Different Data Sources

Dataset I was produced on an HPLC-PDA system, modeling the type of analytical system used in a typical organic chemistry lab. Dataset II was directly read on a UV-Vis spectrophotometer, representing a faster data collection methodology, but without the chromatographic separation afforded by the HPLC-PDA system. Machine learning models trained using Dataset II were also found to provide accurate predictions, suggesting that the presently disclosed subject matter is widely applicable to a variety of different detection methods. As with Dataset I, the median RMSE was comparable using the 2048-bit ECFP6 descriptor or the 1024-bit ECFP descriptor. See Table 4, below. Using either descriptor, the median RMSE of the predictions was substantially lower than predictions using the model trained with Dataset I, (0.06-0.08 vs 0.17). The average median RMSE for predictions made with models trained with three randomly scrambled sets from Dataset II was 0.1, also substantially lower than the scrambled RMSE for Dataset I, which was 0.25.

TABLE 4
Summary of Dataset II training set (1552) and test set (342) information
for UV-Vis spectrum prediction using LSTM or Seq2Seq with attention.
(Scrambled average of N = 3, ±standard deviation).

| | Median | Median | Median | Median RMSE | Median |
|---|---|---|---|---|---|
| Model | RMSE | DTW | R² | derivative | MAE |
| 1024-bit | 0.064 ± | 0.194 ± | 0.710 | 0.008 ± | 0.047 ± |
| ECFP6 | 0.062 | 0.642 | 0.472 | 0.006 | 0.075 |
| 2048-bit | 0.073 ± | 0.196 ± | 0.731 ± | 0.012 ± | 0.046 ± |
| ECFP6 | 0.071 | 0.533 | 0.577 | 0.004 | 0.022 |
| SMILES | 0.078 ± | 0.221 ± | 0.742 ± | 0.012 ± | 0.051 ± |
| | 0.087 | 0.651 | 0.721 | 0.06 | 0.046 |
| Seq2Seq | 0.055 ± | 0.188 ± | 0.699 ± | 0.006 ± | 0.044 ± |
| | 0.071 | 0.431 | 0.259 | 0.007 | 0.052 |
| Scrambled | 0.099 ± | 0.232 ± | 0.593 ± | 0.014 ± | 0.075 ± |
| Ave | 0.091 | 0.813 | .992 | 0.005 | 0.86 |

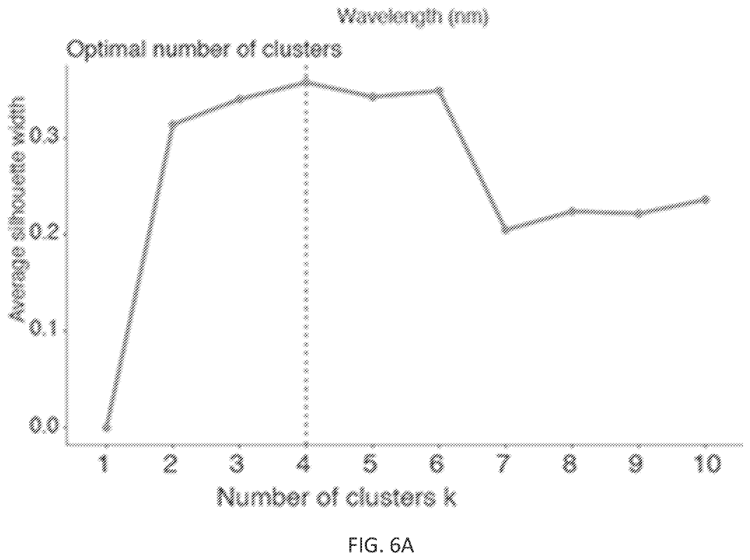

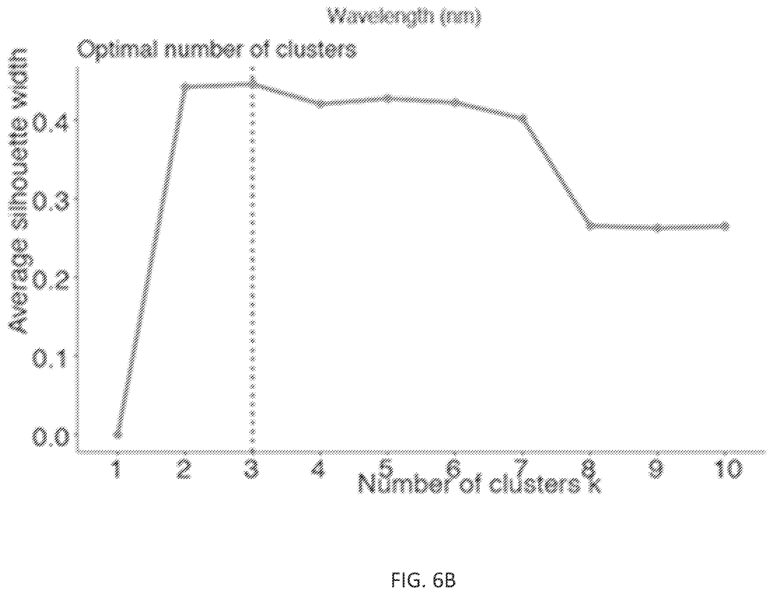

Inspection of the datasets reveals that Dataset II, while derived from a diverse set of compounds, appeared to have a relatively low diversity of spectrum profiles in the training and test sets, with a large number of spectra having few or no features above ˜240 nm. See FIGS. 6A and 6B. To quantify this difference in diversity, the average of the standard deviation of each wavelength value was measured for both datasets. Dataset I had an average standard deviation of 0.23, while Dataset II had an average standard deviation of 0.08, indicating a lower diversity of spectra. Second, shape-based distance was used to divide the spectra into 25 distinct clusters. Dataset I exhibits a higher inter-cluster diversity compared to Dataset II. Using the silhouette method³⁵ to determine the optimal number of clusters, Dataset I is determined to have 4 major clusters, and Dataset II has 3 major spectrum clusters, consistent with the lower spectral diversity of Dataset II. See FIGS. 6A and 6B. Because of the lower spectra diversity, the model trained and tested with Dataset II has a greater statistical probability of predicting the shape of the spectrum when trained with the actual data or the scrambled data. See Table 4, above. This analysis again shows the importance of evaluating the model relative to a scrambled dataset, which captures the overall spectrum diversity for a given dataset. It also confirms that the model is able to learn structure-spectrum relationships for Dataset II. See Table 4, above.

### Comparison of Measures of Prediction Accuracy

It is believed that no single measure of the difference between predicted and actual UV-Vis spectra has been previously adopted as an ideal metric for comparisons. Most comparisons of predicted spectra to measured spectra only consider λₘₐₓ,³⁶ whereas the presently disclosed models predict the entire spectrum over a wavelength range. Therefore, a series of quality metrics were used to evaluate the predictions of the presently disclosed subject matter. In addition to RMSE, other commonly applied metrics are R² and Mean Absolute Error (MAE). Applied to Dataset I, Median R² was similar for 1024-bit ECFP6 and SMILES representations (˜0.63) and lowest for the scrambled average (0.12). Median MAE was lowest for SMILES (0.10) and increased to 0.17 for the scrambled average for Dataset I. See Table 3, above. A similar trend was observed using Dataset II, with stronger measures of concordance for both authentic and scrambled data. See Table 4, above.

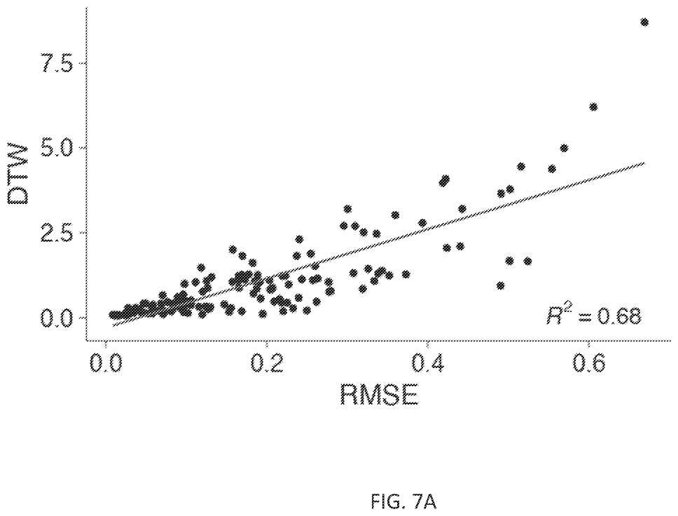

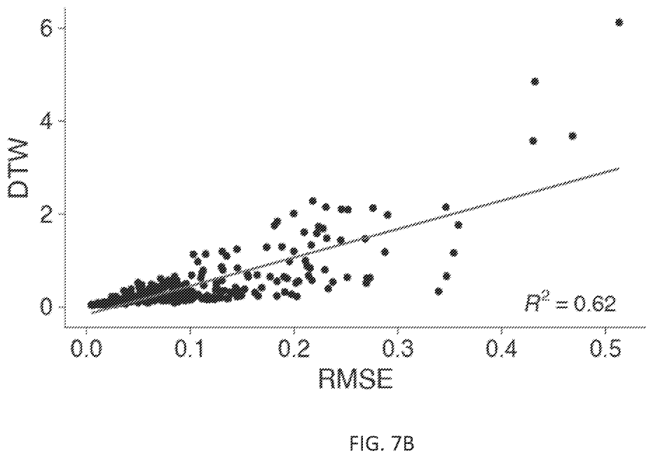

In addition, novel metrics aimed at emphasizing correct prediction of key features of a spectrum were applied. DTW is an approach for comparing data series by finding the optimal match between the series. Applied to spectra, it allows comparison of spectrum shapes when features of the compared spectra are shifted in wavelength.¹³ Thus, in principle, DTW is more robust than measures such as RMSE for comparing spectrum shapes and could also be used for shape-based classification.³⁷ DTW were generated for the test spectra in each dataset and were found to correlate with RMSE (R²>0.6). See FIGS. 7A and 7B. DTW therefore provides an interpretable method to compare predicted and observed spectra to assess machine learning prediction quality. For Dataset I, the median DTW showed considerable variability between 1024-bit, 2048-bit ECFP6 and SMILES representations (0.71-1.03). See Table 3, above. Similarly, for Dataset II the median DTW shows a similar spread (0.194-0.232) (see Table 4, above) on a narrower scale, suggesting the error is generalizable, and therefore the 1024 bit ECFP6 was selected as the more favorable model in the latter case.

The RMSE was also applied between the derivatives of the predicted and actual spectra to emphasize correct prediction of absorption maxima and minima. See Table 4, above. The derivative is obtained using a forward finite-difference approximation applied to wave value at each wavelength:

$${\dot{x}}_{t} = \frac{{x}_{t} - {x}_{t - 1}}{δ t}$$

where xₜ is value of the current wavelength, xₜ₋₁ is the value of the next nm wavelength measured, and δt is the difference between the two wavelengths (in the present case, 1 nm for all spectra wavelength increments).

This measure was the lowest for the 1024-bit ECFP6 and highest for SMILES in Dataset I while being intermediate for the scrambled data. In contrast, SMILES and 2048-bit ECFP6 showed comparable RMSE. SMILES is an end-to-end model; using the encoded SMILES string as an input, whereas ECFP6 are features calculated from the molecule. It is possible that the end-to-end learning of SMILES, while requiring more data, is capable of learning a similar feature representation as fingerprints given a large enough dataset

### Spectrum Predictions for Additional Compounds

After all model test sets were used for evaluation, a prediction was performed on a 17-compound external test set. Though there was no overlap of these compounds with Dataset I, 8 of the 17 were found in Dataset II. Therefore, predictions were made only using the model built with Dataset I. Similar to the test set, both the LSTM model trained with ECFP6 (1024) and the Seq2Seq model had comparable median RMSE (see Table 5, below) for Dataset III. Both models had a higher RMSE and lower Median R² than the test or Dataset III which might be explained by the 17 compounds containing a variety of spectral shapes in comparison to the training, test, and validation sets, which had a number of similar spectrum peaks.

TABLE 5
Summary of Dataset I external UV-Vis spectrum prediction
on 17 compounds in dataset using LSTM or Seq2Seq with
attention. (median value ± standard deviation).

| | Median | Median | Median | Median RMSE | Median |
|---|---|---|---|---|---|
| Model | RMSE | DTW | R² | derivative | MAE |
| 1024-bit | 0.224 ± | 0.872 ± | 0.295 ± | 0.017 ± | 0.139 ± |
| ECFP6 | 0.110 | 0.912 | 0.525 | 0.089 | 0.089 |
| Seq2Seq | 0.236 ± | 0.574 ± | 0.215 ± | 0.020 ± | 0.173 ± |
| SMILES | 0.111 | 0.748 | 0.771 | 0.017 | 0.081 |

### Method Predictions and Molecule Similarity to Training Set

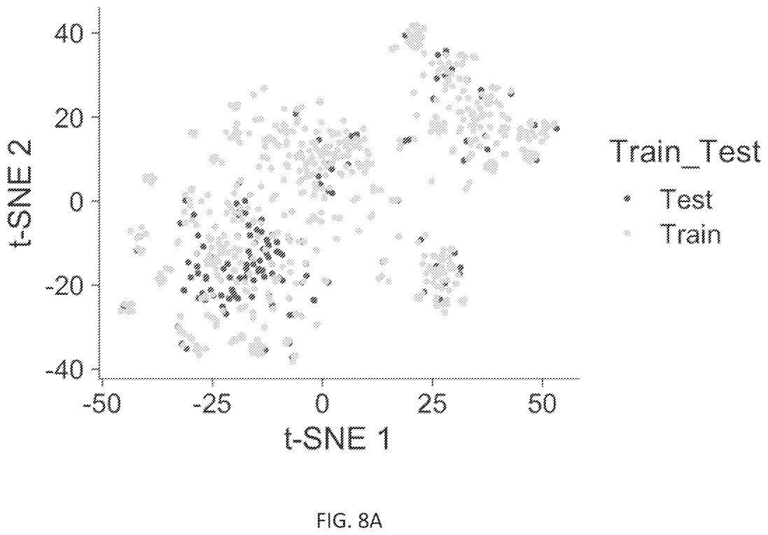

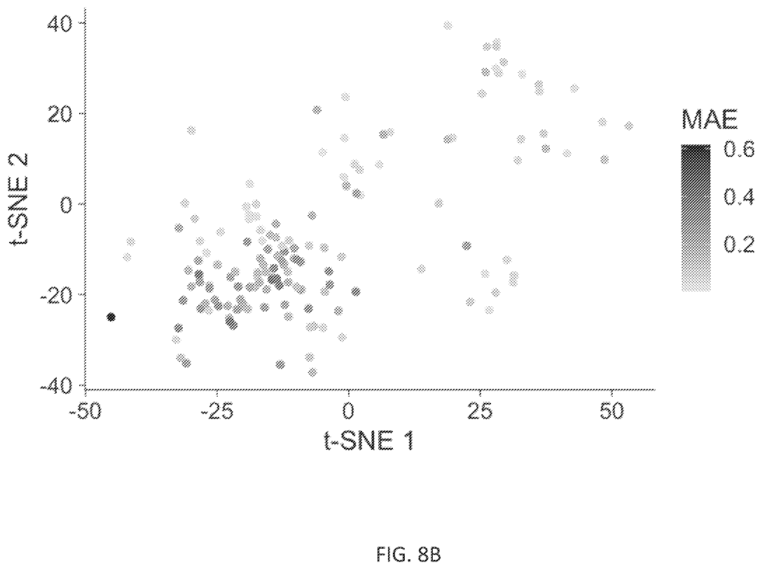

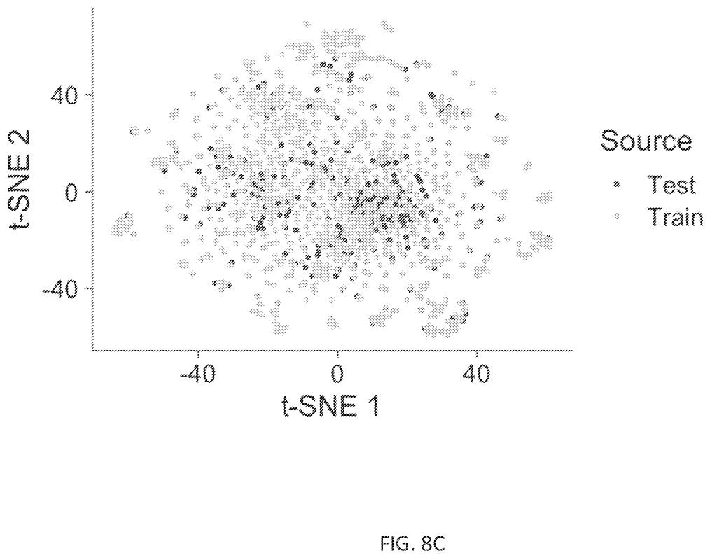

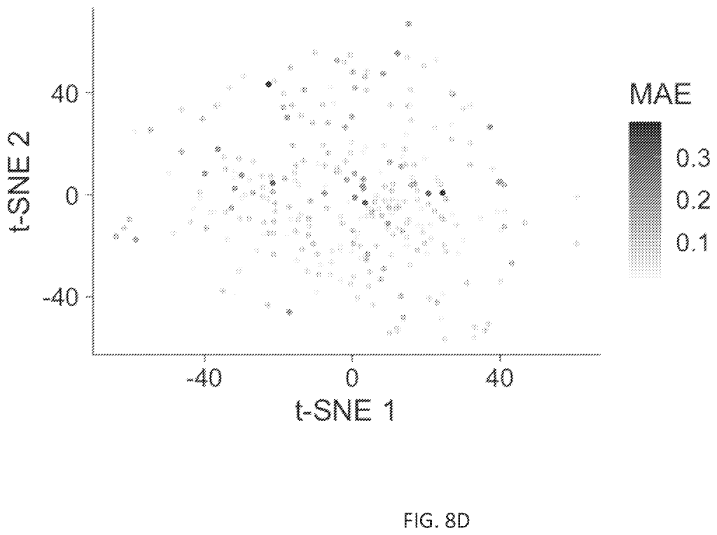

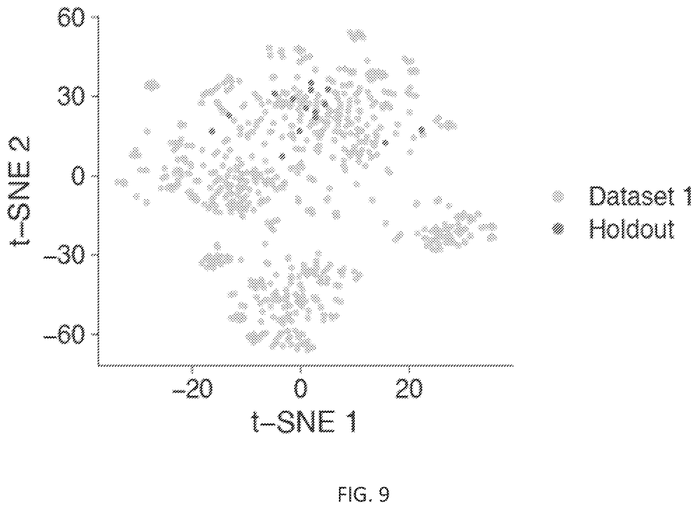

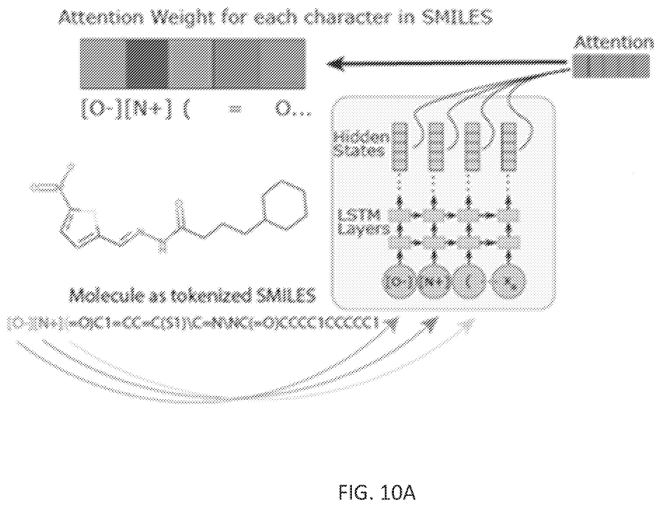

Chemical space is infinite.³⁸ Therefore, it would be unexpected for machine learning models trained with hundreds to thousands of molecules to correctly predict a UV-Vis spectrum for all possible new molecules. The presently disclosed method was capable of predicting near-identical spectral curves for some compounds but missed important features for others. The t-distributed stochastic neighbor embedding (t-SNE) plots³⁹ of structural similarity (based on ECFP6 fingerprints) suggests that predictive power is determined by training and test set overlap. Where the density of training examples is sparse in relation to the density of the test examples, the MAE of predictions is generally higher. See FIGS. 8A-8D. This observation suggests that the reliability of predictions can be improved with sufficient representation in the training set of the model. The additional compounds (Dataset III) were also well distributed in the t-SNE plot for the Dataset I (see FIG. 9) suggesting they were likely within the applicability domain of this model.

### Evaluating Chemical-Substructure Contributions to Spectrum Prediction by Exploiting Model Attention Weights

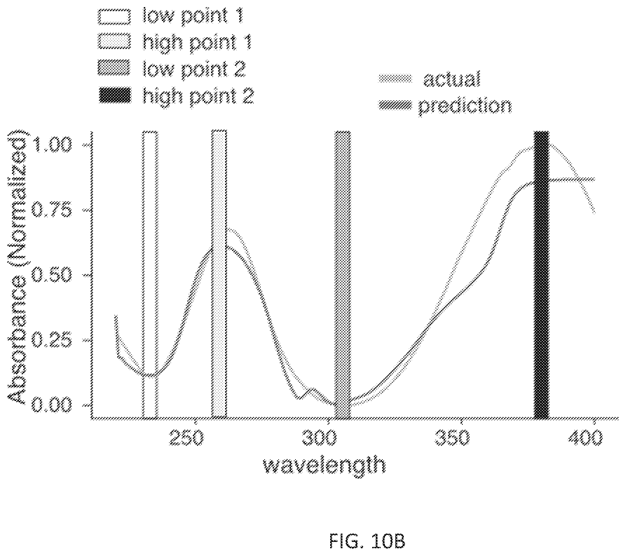

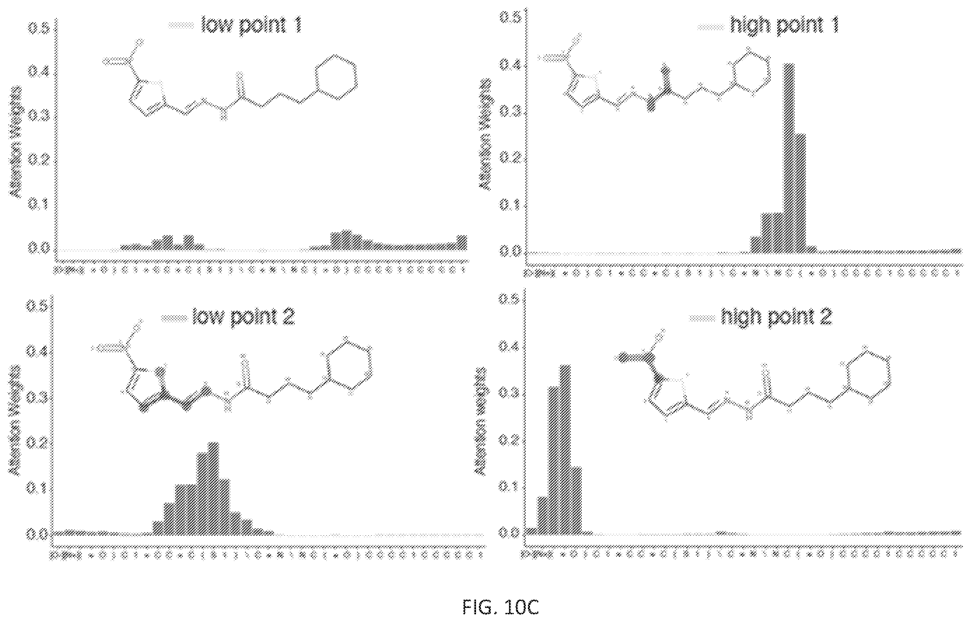

One of the advantages of using a Seq2Seq model with attention is the ability to visualize the attention mechanism.³² In the presently disclosed Seq2Seq model, compounds are represented as tokenized SMILES strings. Upon generation of each wavelength value, a corresponding vector of weights over each character in the input is generated. See FIG. 10A. This vector of weights describes what parts of the input the model is “paying attention to” at each prediction step. Although caution can be used to not make direct inference from attention alone, this mechanism can be exploited to observe what part the compound structure the model is paying attention to and derive substructure importance from UV-Vis spectra. A spectrum was chosen that was predicted with reasonable accuracy for an example. See FIG. 10B. Here, two “low points” and two “high points” were selected and the attention weights were observed for each. At the lowest wavelengths, the model's attention is not focused on any part of the input. See FIG. 10C, top-left. During the first peak, however, the model is focused on the amide group. The second low point on the spectrum shows a focus on the thiophene ring, and the λₘₐₓ indicates attention focus on the nitro group. This type of structure-spectrum analysis can also inform efforts to develop rules to calculate the λₘₐₓ based on substructure features.^{40,41}

In practice, UV-Vis spectra are most commonly used in reference to specific qualified standards or spectral libraries. The theoretical prediction of spectra has not achieved sufficient accuracy for routine use in chemistry labs, particularly for chemists analyzing mixtures of crude reaction products or extractions. In contrast, predictive tools for NMR and FT-IR spectra are used by almost all synthetic chemists in identification, characterization and structural elucidation of novel compounds (e.g. NMR predictor software, ACDlabs).⁴² Given that chemists routinely collect UV-Vis spectral data as part of standard HPLC analysis workflows, these data are essentially “free” and underutilized by them. The ability to accurately predict UV-vis spectra de novo would provide for chemists to more easily identify compounds of interest without the need for qualified reference standards.

The most commonly used method to date for UV-Vis spectrum prediction is TD-DFT (see Table 6, below) using CAM-B3LYP functionals.¹⁷ This approach requires quantum chemistry software, significant computing resources, and expertise in their use and interpretation. Nevertheless, it has been used in hundreds of publications for diverse range of compounds. Most of these publications report studies of individual compounds or at most a few analogs, and the experimental data for the various studies have been generated in a variety of solvents, limiting their value as a spectrum database. Most measure agreement between prediction and experiment only at λₘₐₓ, providing at best, a qualitative assessment of agreement for other spectral features. In many cases, the predicted values of λₘₐₓ are significantly different than those observed.

TABLE 6
Examples of diverse small molecules described in the literature
with experimental and predicted UV-Vis spectra using DFT.

| Molecule | Application | Prediction Methods | Summary | Ref. |
|---|---|---|---|---|
| Carmoisine | Azo dye, food | DFT using hybrid | excited state 1 calc. | 51 |
| | additive | B3PW91 with | 488 nm (exp 510 nm), | |
| | | LANL2DZ | excited state 3 calc. | |
| | | | 366 (exp 335 nm) | |
| Chalcone (E)-3-mesityl-1- | Pharmaceutical | DFT using B3LYP | Agreement in | 52 |
| (napthylen-2-yl) prop-2-en- | | and 6-311G (d, p) | prediction of four | |
| 1-one | | basis set | electronic | |
| | | | transition bands at | |
| | | | 216, 258, 302 and | |
| | | | 321 nm | |
| 2-(bromoacetyl)-benzo(b)furan | Pharmaceutical | DFT using B3LYP | Predicted one peak | 53 |
| | | and 6-31G (d, p) | at 344.6 nm (exp | |
| | | basis set | 353 nm) | |
| Disperse red 1 acrylate | Azo dye | DFT using B3LYP | predicted max | 54 |
| | | and 6-311G (d, p) | absorption at 397 | |
| | | basis set | (exp 472 nm in | |
| | | | methanol) | |
| Pistagremic acid | Natural product | DFT using B3LYP | predicted max | 55 |
| | with pharmaceutical | and 6-31G (d, p) | excitation energy at | |
| | uses | basis set | 229 nm (exp 265 | |
| | | | nm in chloroform) | |
| PAZB-3, PAZB-11, PAZB-12 | Azo dyes | DFT multiple | ⅓ agreement | 56 |
| | | methods including | PAZB-3 calculated | |
| | | PBE1PBE with | lamda max = 425 | |
| | | 6-31 + G basis | nm (exp 371 nm in | |
| | | set | DMF), PAZB-11 | |
| | | | 372 nm (exp 375 | |
| | | | nm), PAZB-12 344 | |
| | | | nm (exp 377 nm) | |
| Indigo analogs 7a, 7b, 7c, 7d | Dyes | DFT using B3LYP | Standard error | 57 |
| | | and 6-311G (d, p) | range 8.3-12.3 in | |
| | | basis set | lamda max. 6 | |
| | | | solvents tested | |
| | | | experimentally | |
| Imatinib | pharmaceutical | DFT using B3LYP | various pH and | 58 |
| | | and 6-311G (d, p) | solvents were | |
| | | basis set | tested while DFT | |
| | | | used gas phase | |
| | | | calculations lamda | |
| | | | max = 240, 255 and | |
| | | | 284 nm (exp 234- | |
| | | | 243 nm and 259- | |
| | | | 268 nm in water | |
| | | | and 228-238 nm | |
| | | | and 270-274 nm in | |
| | | | ethanol) | |
| 4,5-dimethyl-o- | Pharmaceuticals, | DFT using B3LYP | calculated lamda | 59 |
| phenylenediamine | dyes, plastics | and 6-311G (d, p) | max at 237, 242, | |
| | | basis set | 293 nm (exp 210, | |
| | | | 240 and 300 nm in | |
| | | | ethanol) | |
| Trimethoprim | Pharmaceutical | DFT using B3LYP | lamda max 271.6 | 60 |
| | | and 6-311G (d, p) | nm (exp 278 nm) | |
| | | basis set | | |
| S-bromo-2- | Pharmaceutical | DFT using B3LYP | Lamda max 275.1 | 61 |
| ethoxyphenylboronic acid | | and 6-311G (d, p) | and 227.6 nm (exp | |
| | | basis set | 290.8 and 230.2 in | |
| | | | ethanol) spectra are | |
| | | | very different | |
| 4-hexyloxy-3- | Pharmaceutical | DFT using B3LYP | Lamda max 314.4, | 62 |
| methoxybenzaldehyde | | and 6-311G (d, p) | 271.4 and 231.1 nm | |
| | | basis set | (exp 308, 276 and | |
| | | | 230 nm) although | |
| | | | spectra look | |
| | | | different | |
| Cinnamates | UV filters | Used 6 different | B3LYP, B3P86 had | 6 |
| | | functionals and | lamda max of 320. | |
| | | 6-311G (d, p) | and 317.27 nm | |
| | | basis set | respectively (exp | |
| | | | 309 nm) for | |
| | | | ethylhexyl | |
| | | | methoxycinnamate. | |
| | | | B3LYP, B3P86 had | |
| | | | lamda max of | |
| | | | 321.54 and 319.42 | |
| | | | nm respectively | |
| | | | (exp 310 nm) | |
| | | | Isoamyl p- | |
| | | | methoxycinnamate | |
| 4-butyl benzoic acid | Pharmaceuticals | DFT using B3LYP | Lamda max 245.8 | 63 |
| | | and 6-311G (d, p) | nm (exp 237 nm in | |
| | | basis set | ethanol) and 245.5 | |
| | | | (exp 238 nm in | |
| | | | water) | |
| 3-fluorophenylboronic acid | Pharmaceuticals, | DFT using B3LYP | Lamda max 219.7 | 64 |
| | catalysts | and 6-311G (d, p) | and 248.2 nm (exp | |
| | | basis set | 217.2 and 268.9 nm | |
| | | | in ethanol) and | |
| | | | 219.5 and 248.1 | |
| | | | (exp 216.1 and | |
| | | | 269.7 nm in water) | |
| Difluoroboron b-diketonates | Dyes | DFT using B3LYP | 7 dyes were | 65 |
| | | and 6-311G (d, p) | compared with the | |
| | | basis set | following predicted | |
| | | | lamda max | |
| | | | 324.6 (exp 303.0 | |
| | | | nm), 286.0 (exp | |
| | | | 274.0 nm), 241.6 | |
| | | | (exp 251.3 nm), | |
| | | | 276.9 (266.0 nm), | |
| | | | 192.8 (exp 214.1 | |
| | | | nm), 265.2 (exp | |
| | | | 256.4 nm) 235.5 | |
| | | | (exp 237.5) | |
| Squamocin | Natural product | DFT using B3LYP | Lamda max 230 | 66 |
| | with pharmaceutical | and 6-31G (d, p) | and 288 nm (exp | |
| | applications | basis set | 213 nm) | |

Though the limitations of purely theoretical approaches to predicting UV-Vis spectra hinder the application of these approaches to compound identification and characterization in organic chemistry, chemists routinely use empirical rules to make qualitative or partial predictions of compounds' UV-Vis absorbance behavior. The utility of such methods suggests the potential for data-driven approaches such as machine learning to prediction of UV-Vis spectra. Key issues that have been addressed by the presently disclosed subject matter to realize this objective are the availability of sufficient data for training, validating, and testing machine learning (ML) model algorithms; the relationship between the content of training data and the reliability of predictions; machine readable (i.e., vector) representations of molecular structure that capture sufficient detail to generalize structure-spectrum relationships; network architectures that output predicted spectra that are continuous across a wavelength range; and useful metrics for assessing the predictive power of ML models.

Despite the routine nature of UV-Vis spectrum acquisition, assembly of a sizeable dataset from publicly available sources that meets the needs of training and testing for spectrum prediction was not possible. Existing publicly available datasets are inadequate because they lack full spectra across a consistent wavelength range (rather than Imax only or varying wavelength ranges), absorption values across the wavelength range (rather than plotted spectra only), consistent solvent environments (solvent composition and pH), or a diversity of molecular structures (e.g. the compound sets often being focused on an analogous series of compounds such as dyes). Data harvested piecewise from the literature suffered similar deficits. These limitations can be avoided by constructing new datasets.

The library of compounds used to construct Dataset I included an internal collection aggregated from a variety of projects with a range of objectives undertaken at SRI International (Menlo Park, California, United States of America). In addition to emulating the type of analytical system used in a typical organic chemistry lab, the HPLC methodology that was used for collection of Dataset I ensured that the spectra analyzed were of pure compounds. The larger library of compounds in Dataset II from a commercial vendor comprised a wide range of drugs and natural products. By using these two datasets, machine learning models have been created that relate to a broader range of compound classes than literature datasets created primarily using dyes.

At the outset, it was unknown how much data would be needed for machine learning models to learn structure-UV-VIS spectrum relationships to generalize to new molecules. It was found that surprisingly small datasets can result in accurate predictions for new molecules. With less than 1000 molecules, good levels of accuracy of prediction can be obtained as judged by median statistics. Not surprisingly, it appears that the quality of spectrum prediction depends on the overlap between the chemical space of the training data and the compounds for which predictions are made. See FIGS. 10A-10C. Similarly, the accuracy of predictions was found to depend on the similarity of spectral profiles between the training compounds and the compounds for prediction. Expanded datasets that cover more chemical space can be used to improve the reliability of predictions. Models for different solvent conditions can also be assessed to determine if datasets can be extended to create “generic UV-Vis spectrum models.”

The LSTM network architectures employed according to the presently disclosed subject matter are well-suited to the modeling of UV-Vis spectra. The recurrent structure of the LSTM architecture facilitates the modeling of spectra as continuous data series. Such models are particularly apt for UV-Vis spectra, which are typically smooth functions with broad features. The LSTM models described can be generated in minutes, and molecule predictions are processed in seconds. The Seq2Seq model with attention provided predictive accuracy comparable to the LSTM model that we tested and represents a novel method for visualizing what parts of the chemical substructure are most relevant to the prediction at hand. It is believed that the presently disclosed subject matter represents the first use of an attention mechanism for probing substructure-UV-Vis prediction relevance. Without being bound to any one theory, attention placed on certain substructures that appear repeatedly for specific wavelength peaks can indicate a chemical feature to investigate further and could the focus of further research. Without being bound to any one theory, it is believe that the attention placed on each atom can be interpreted as relevant to the predictive ability and this information can be used to refine the model by altering the training set.

It is demonstrated herein that UV-Vis spectra can be predicted from molecular structure alone (i.e., without additional physics-based information) represented by either ECFP6 descriptors or SMILES. It has been previously demonstrated that compression of 1024-bit fingerprints to 8 bits of information could facilitate the use of machine learning approaches on a quantum computer⁴³ and, without being bound to any one theory, it was hypothesized that this same approach could be used to assess how much structural information needed to be retained for accurate model spectra prediction. The reduction to 1024-bit fingerprints did not result in any significant information loss versus the 2048-it fingerprints, while ECFP6 8-bit compression showed a dramatic loss based on degradation of statistical measures such as RMSE. See Table 3.

Development of models to predict UV-Vis spectra uses metrics to evaluate the quality of predictions. Statistical measures such as RMSE, R², and MAE are commonly used metrics of agreement between predicted and actual values, and have been applied to evaluate the models described herein, to test different input formats, and to test the effect of scrambling the structure-spectrum relationship during training. MAE has been used as the metric of loss during training of the models. It was found that these measures are generally in concurrence. To the extent they differ, RMSE agrees best with the qualitative assessment of prediction quality.

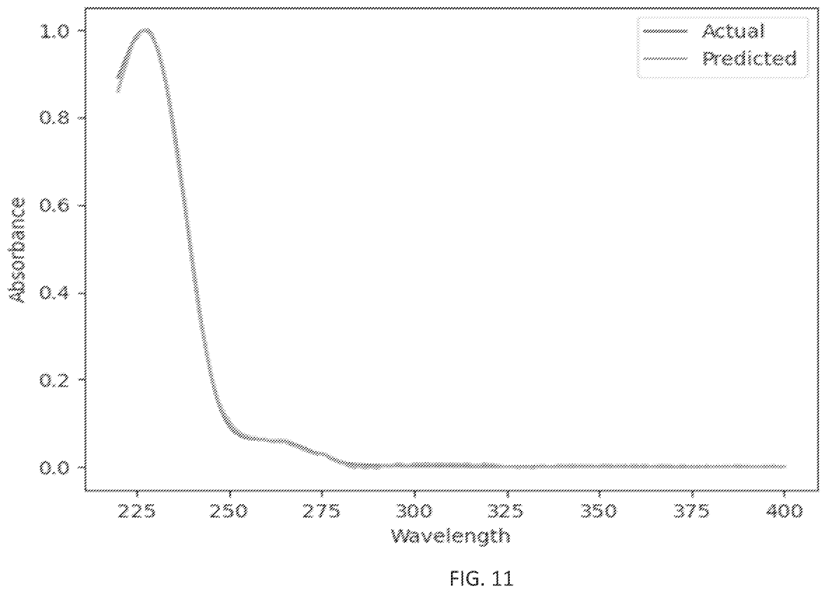

Many test set predictions were remarkably close to the observed spectra (e.g., spectra in FIGS. 5A, 5B, and 11), an agreement reflected in values for RMSE, R², and MAE. However, other spectrum predictions of the models capture important and useful features of the observed spectra in ways that are not well-reflected in these common statistical measures. For example, a small shift in wavelength of a large absorption peak results in a large contribution to RMSE but will often have a small impact on the utility of the prediction for distinguishing between two compounds. Similarly, a discrepancy in the relative height of a peak in a predicted spectrum from an actual spectrum can degrade the RMSE but have a small impact on interpretation. To address this shortcoming of standard statistical measures, additional measures of prediction quality have been applied to the models, DTW and derivative spectrum RMSE.

DTW is a distance measure technique that allows a non-linear mapping between two signals by minimizing the distance between them.¹¹ This method is flexible, allowing two data series that are similar but locally out of phase to align non-linearly. It is a well-known solution for time-series alignment.¹² It is believed that DTW has not been used previously for comparisons of spectra. As a measure of agreement between predicted and actual spectra, it accommodates small shifts in wavelength between spectra of similar shape DTW is correlated with RMSE for the predictions made with the test sets. See FIGS. 7A and 7B. Median DTW is also correlated with median RMSE. See Tables 3 and 4.

Comparison of derivatives of predicted and observed spectra also allows comparison of the overall shapes of spectra, emphasizing agreement in the wavelength positions of peaks and valleys, where the value of the derivative is zero irrespective of the magnitude of the absorption at those wavelengths. Derivative spectroscopy is frequently used to visualize poorly resolved spectral features and to differentiate similar spectra.⁴⁴ However, it is not believed to have been previously used in quantitative comparison of predicted and experimental spectra. As with DTW, the trend in median values for this measure mirrors that of median RMSE.

Functional tests for assessing the quality of spectrum predictions provide a practical and intuitive measure of predictive success. The test described herein, correspondence of peak wavelengths between predicted and experimental spectra, emphasizes the peak positions over other spectrum features. Though this approach is similar to the typical analysis of results of TD-DFT predictions, which judges success by prediction of λₘₐₓ values only, the measure applied herein adds the rigor of requiring that no peaks are predicted that are not in the actual spectrum.

In summary, the machine learning technique embodied in the presently disclosed subject matter provides for very large compound libraries to be scored more quickly than previous methods. Thus, it can provide chemists the ability to more rapidly and reliably identify compounds with desirable UV-Vis spectra. It can have applications for new compound discovery (e.g. prediction of dye colors), organic chemistry reaction monitoring, phototoxicity prediction, and numerous other important chemistry applications.²⁻⁹ Alternative spectrum comparison measures such as DTW have been shown to help in assessment of observed and predicted spectra. These scores can be used in the future as elements of machine learning model cost functions. Comparison of 2D and 3D descriptors, evaluation and optimization of additional machine learning models,¹⁴⁻¹⁶ as well as applying additional models for selection of training and test sets can be performed. The models used herein are also likely applicable to nuclear magnetic resonance (NMR) and mass spectroscopy (MS) spectrum prediction. Generation of spectra for significantly larger training sets (tens to hundreds of thousands of molecules) can assist in broadening the scope of these computational models and be useful in training recurrent neural network models to assist in the de novo design of molecules^{45,46} with a particular spectrum of interest for specific applications requiring ideal physicochemical or UV-Vis properties.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1. Schmidt, F.; Wenzel, J.; Halland, N.; Gussregen, S.; Delafoy, L.; Czich, A., Computational Investigation of Drug Phototoxicity: Photosafety Assessment, Photo-Toxophore Identification, and Machine Learning. Chem Res Toxicol 2019, 32 (11), 2338-2352. DOI: 10.1021/acs.chemrestox.9b00338.
2. Ghidinelli, S.; Longhi, G.; Abbate, S.; Hattig, C.; Coriani, S., Magnetic Circular Dichroism of Naphthalene Derivatives: A Coupled Cluster Singles and Approximate Doubles and Time-Dependent Density Functional Theory Study. J Phys Chem A 2020. DOI: 10.1021/acs.jpca.0c09669.
3. Anouar el, H.; Weber, J. F., Time-dependent density functional theory study of UV/vis spectra of natural styrylpyrones. Spectrochim Acta A Mol Biomol Spectrosc 2013, 115, 675-82. DOI: 10.1016/j.saa.2013.06.114.
4. Martynov, A. G.; Mack, J.; May, A. K.; Nyokong, T.; Gorbunova, Y. G.; Tsivadze, A. Y., Methodological Survey of Simplified TD-DFT Methods for Fast and Accurate Interpretation of UV-Vis-NIR Spectra of Phthalocyanines. ACS Omega 2019, 4 (4), 7265-7284. DOI: 10.1021/acsomega.8b03500.
5. Daengngern, R.; Camacho, C.; Kungwan, N.; Irle, S., Theoretical Prediction and Analysis of the UV/Visible Absorption and Emission Spectra of Chiral Carbon Nanorings. J Phys Chem A 2018, 122 (37), 7284-7292. DOI: 10.1021/acs.jpca.8b07270.
6. Garcia, R. D.; Maltarollo, V. G.; Honorio, K. M.; Trossini, G. H., Benchmark studies of UV-vis spectra simulation for cinnamates with UV filter profile. J Mol Model 2015, 21 (6), 150. DOI: 10.1007/s00894-015-2689-y.
7. Aguilar-Martinez, M.; Cuevas, G.; Jimenez-Estrada, M.; Gonzalez, I.; Lotina-Hennsen, B.; Macias-Ruvalcaba, N., An Experimental and Theoretical Study of the Substituent Effects on the Redox Properties of 2-[(R-phenyl)amine]-1,4-naphthalenediones in Acetonitrile. J Org Chem 1999, 64 (10), 3684-3694. DOI: 10.1021/jo9901860.
8 Blase, X.; Duchemin, I.; Jacquemin, D.; Loos, P. F., The Bethe-Salpeter Equation Formalism: From Physics to Chemistry. J Phys Chem Lett 2020, 11 (17), 7371-7382. DOI: 10.1021/acs.jpclett.0c01875.
9 Yahyaei, H.; Shahab, S.; Sheikhi, M.; Filippovich, L.; Almodarresiyeh, H. A.; Kumar, R.; Dikusar, E.; Borzehandani, M. Y.; Alnajjar, R., Anisotropy (optical, electrical and thermal conductivity) in thin polarizing films for UV/Vis regions of spectrum: Experimental and theoretical investigations. Spectrochim Acta A Mol Biomol Spectrosc 2018, 192, 343-360. DOI: 10.1016/j.saa.2017.11.029.
10. Greff, K.; Srivastava, R. K.; Koutnik, J.; Steunebrink, B. R.; Schmidhuber, J., LSTM: A Search Space Odyssey. IEEE Trans Neural Netw Learn Syst 2017, 28 (10), 2222-2232. DOI:

10.1109/TNNLS.2016.2582924.
11. Berndt, D.; Clifford, J. In Using dynamic time warping to find patterns in time series, AAAI Workshop on Knowledge Discovery in Databases, 1994; pp 229-248.
12. Bagnall, A.; Lines, J.; Bostrom, A.; Large, J.; Keogh, E., The Great Time Series Classification Bake off: A Review and Experimental Evaluation of Recent Algorithmic Advances. Data Mining and Knowledge Discovery 2017, 31, 606-660.
13. Keogh, E.; Ratanamahatana, C. A., Exact indexing of dynamic time warping. Knowledge and Information Systems 2004, 7, 358-386.
14. Lane, T.; Russo, D. P.; Zorn, K. M.; Clark, A. M.; Korotcov, A.; Tkachenko, V.; Reynolds, R. C.; Perryman, A. L.; Freundlich, J. S.; Ekins, S., Comparing and Validating Machine Learning Models for Mycobacterium tuberculosis Drug Discovery. Mol Pharm 2018, 15 (10), 4346-4360. DOI: 10.1021/acs.molpharmaceut.8b00083.
15. Russo, D. P.; Zorn, K. M.; Clark, A. M.; Zhu, H.; Ekins, S., Comparing Multiple 15. Machine Learning Algorithms and Metrics for Estrogen Receptor Binding Prediction. Mol Pharm 2018, 15 (10), 4361-4370. DOI: 10.1021/acs.molpharmaceut.8b00546.
16. Zorn, K. M.; Lane, T. R.; Russo, D. P.; Clark, A. M.; Makarov, V.; Ekins, S., Multiple Machine Learning Comparisons of HIV Cell-based and Reverse Transcriptase Data Sets. Mol Pharm 2019, 16 (4), 1620-1632. DOI: 10.1021/acs.molpharmaceut.8b01297.
17. Gonzalez, L.; Escudero, D.; Serrano-Andres, L., Progress and challenges in the calculation of electronic excited states. Chemphyschem 2012, 13 (1), 28-51.
18. Perkampus, H. H., UV-VIS Spectroscopy and Its Applications. Springer-Verlag: Berlin, 1992.
19. Shen, Y.; Abolhasani, M.; Chen, Y.; Xie, L.; Yang, L., Coley, C. W.; Bawendi, M. G.; Jensen, K. F., In-Situ Microfluidic Study of Biphasic Nanocrystal Ligand-Exchange Reactions Using an Oscillatory Flow Reactor. Angew Chem Int Ed Engl 2017, 56 (51), 16333-16337.
20. Simeonov, A.; Davis, M. I., Interference with Fluorescence and Absorbance. In Assay Guidance Manual, Markossian, S.; Sittampalam, G. S., Grossman, A.; Brimacombe, K.; Arkin, M.; Auld, D.; Austin, C. P.; Baell, J.; Caaveiro, J. M. M.; Chung, T. D. Y.; Coussens, N. P.; Dahlin, J. L.; Devanaryan, V.: Foley, T. L.; Glicksman, M.; Hall, M. D.; Haas, J. V.; Hoare, S. R. J.; Inglese, J.; Iversen, P. W.; Kahl, S. D.; Kales, S. C.; Kirshner, S.; Lal-Nag, M.; Li, Z.; McGee, J.; McManus, O.; Riss, T.; Saradjian, P.; Trask, O. J., Jr.; Weidner, J. R.; Wildey, M. J.; Xia, M.; Xu, X., Eds. Bethesda (MD), 2004.
21. Simine, L.; Allen, T. C.; Rossky, P. J., Predicting optical spectra for optoelectronic polymers using coarse-grained models and recurrent neural networks. Proc Natl Acad Sci USA 2020, 117 (25), 13945-13948.
22. Kuenemann, M. A.; Szymczyk, M.; Chen, Y.; Sultana, N.; Hinks, D., Freeman, H. S.; Williams, A. J.; Fourches, D.; Vinueza, N. R., Weaver's historic accessible collection of synthetic dyes: a cheminformatics analysis. Chem Sci 2017, 8 (6), 4334-4339.
23. Talrose, V.; Yermakov, A. N., Leskin, A. N.; Usov, A. A.; Goncharova, A. A.; Messineva, N. A.; Usova, N. V.; Efimkina, M. V.; Aristova, E. V. NIST chemistry webbook; online at webbook.nist.gov/chemistry/uv-vis/.
24. Taniguchi, M.; Du, H.; Lindsey, J. S., PhotochemCAD 3: Diverse Modules for Photophysical Calculations with Multiple Spectral Databases. Photochem Photobiol 2018, 94 (2), 277-289.
25. Noelle, A.; Vandaele, A. C.; Martin-Torres, J.; Yuan, C.; Rajasekhar, B. N.; Fahr, A.; Hartmann, G. K.; Lary, D.; Lee, Y. P.; Limao-Vieira, P.; Locht, R.; McNeill, K.; Orlando, J. J.; Salama, F.; Wayne, R. P., UV/Vis(+) photochemistry database: Structure, content and applications. J Quant Spectrosc Radiat Transf 2020, 253.
26. Venkatraman, V.; Raju, R.; Oikonomopoulos, S. P.; Alsberg, B. K., The dye-sensitized solar cell database. J Cheminform 2018, 10 (1), 18.
27. Beard, E. J.; Sivaraman, G.; Vazquez-Mayagoitia, A.; Vishwanath, V.; Cole, J. M., Comparative dataset of experimental and computational attributes of UV/vis absorption spectra. Sci Data 2019, 6 (1), 307.
28. Anon KnowItAll UV-Vis Spectral Database Collection; available online at sciencesolutions.wiley.com/solutions/technique/uv-vis/knowitall-vu-vis-collection/.
29. Dalby, A.; Nourse, J. G.; Hounshell, W. D.; Gushurst, A. K. I.; Grier, D. L.; Leland, B. A.; Laufer, J., Description of several chemical structure file formats used by computer programs developed at Molecular Design Limited. Journal of Chemical Information and Computer Sciences 1992, 32 (3), 244-255.
30. Weininger, D., SMILES, a chemical language and information system. 1. Introduction to methodology and encoding rules. Journal of Chemical Information and Computer Sciences 1988, 28 (1), 31-36.
31. Sutskever, I.; Vinyals, O.; Le, Q. V., Sequence to Sequence Learning with Neural Networks. arXiv: 1409.3215v3 2014.
32. Luong, T.; Pham, H. T.; Manning, C. D. In Effective Approaches to Attention-based Neural Machine Translation, Proceedings of the 2015 Conference on Empirical Methods in Natural Language Processing, Lisbon, Portugal, Lisbon, Portugal, 2015; pp 1412-1421.
33. Rogers, D.; Brown, R. D.; Hahn, M., Using extended-connectivity fingerprints with Laplacian-modified Bayesian analysis in high-throughput screening follow-up. J Biomol Screen 2005, 10 (7), 682-6.
34. Rogers, D.; Hahn, M., Extended-connectivity fingerprints. J Chem Inf Model 2010, 50 (5), 742-54.
35. Rousseeuw, P. J., Silhouettes: A graphical aid to the interpretation and validation of cluster analysis. Journal of Computational and Applied Mathematics 1987, 20, 53-65.
36. Shao, Y.; Mei, Y.; Sundholm, D.; Kaila, V. R. I., Benchmarking the Performance of Time-Dependent Density Functional Theory Methods on Biochromophores. Journal of Chemical Theory and Computation 2020, 16 (1), 587-600.
37. Wallwitz, R.; Baumann, W., A new shape-oriented classification method for UV/VIS-spectra. Anal Bioanal Chem 1996, 354 (4), 385-91.
38. Dobson, C. M., Chemical space and biology. Nature 2004, 432 (7019), 824-828.
39. van der Maaten, L.; Hinton, G., Visualizing Data using t-SNE. J Machine Learning Research 2008, 9, 2579-2605.
40. Woodward, R. B., Structure and the Absorption Spectra of α,β-Unsaturated Ketones. Journal of the American Chemical Society 1941, 63 (4), 1123-1126.
41. Fieser, L. F.; Fieser, M.; Rajagopalan, S., ABSORPTION SPECTROSCOPY AND THE STRUCTURES OF THE DIOSTEROLS. The Journal of Organic Chemistry 1948, 13 (6), 800-806.
42. Moser, A.; Elyashberg, M. E.; Williams, A. J.; Blinov, K. A.; Dimartino, J. C., Blind trials of computer-assisted structure elucidation software. J Cheminform 2012, 4 (1), 5.
43. Batra, K., Zom, K. M.; Foil, D. H.; Minerali, E.; Gawriljuk, V. O.; Lane, T. R.; Ekins, S., Quantum Machine Learning Algorithms for Drug Discovery Applications. J Chem Inf Model 2021, 61 (6), 2641-2647.
44. Ojeda, C. B.; Rojas, F. S., Recent developments in derivative ultraviolet/visible absorption spectrophotometry. Anal Chim Acta 2004, 518, 1-24.
45. Segler, M. H. S.; Kogej, T.; Tyrchan, C.; Waller, M. P., Generating Focused Molecule Libraries for Drug Discovery with Recurrent Neural Networks. ACS Cent Sci 2018, 4 (1), 120-131.
46. Gomez-Bombarelli, R.; Wei, J. N., Duvenaud, D.; Hernandez-Lobato, J. M.; Sanchez-Lengeling, B.; Sheberla, D.; Aguilera-Iparraguirre, J.; Hirzel, T. D.; Adams, R. P.; Aspuru-Guzik, A., Automatic Chemical Design Using a Data-Driven Continuous Representation of Molecules. ACS Cent Sci 2018, 4 (2), 268-276
47. Montero, P.; Vilar, J. A., TSclust: An R Package for Time Series Clustering. J Statistical Software 2014, 62.
48. Team, R. C. R: A language and environment for statistical computing, available online at R-project.org.
49. Murrell, B.; Murrell, D.; Murrell, H., Discovering General Multidimensional Associations. PLOS One 2016, 11 (3), e0151551.
50. Rousseeuw, P. J., Silhouettes: A graphical aid to the interpretation and validation of cluster analysis. Journal of Computational and Applied Mathematics 1987, 20, 53-65.
51. Snehalatha, M.; Ravikumar, C.; Hubert Joe, I.; Sekar, N.; Jayakumar, V. S., Spectroscopic analysis and DFT calculations of a food additive carmoisine. Spectrochim Acta A Mol Biomol Spectrosc 2009, 72 (3), 654-62.
52. Barakat, A.; Al-Majid, A. M.; Soliman, S. M.; Mabkhot, Y. N.; Ali, M.; Ghabbour, H. A.; Fun, H. K.; Wadood, A., Structural and spectral investigations of the recently synthesized chalcone (E)-3-mesityl-1-(naphthalen-2-yl) prop-2-en-1-one, a potential chemotherapeutic agent. Chem Cent J 2015, 9, 35.
53. Veeraiah, A., FT-IR, FT-Raman, UV/Vis spectra and fluorescence imaging studies on 2-(bromoacetyl)benzo(b)furan by ab initio DFT calculations. Spectrochim Acta A Mol Biomol Spectrosc 2015, 147, 212-24.
54. Cinar, M.; Coruh, A.; Karabacak, M., FT-IR, UV-vis, 1H and 13C NMR spectra and the equilibrium structure of organic dye molecule disperse red 1 acrylate: a combined experimental and theoretical analysis. Spectrochim Acta A Mol Biomol Spectrosc 2011, 83 (1), 561-9
55. Ullah, H.; Rauf, A.; Ullah, Z., Fazl i, S., Anwar, M.; Shah, A. U.; Uddin, G., Ayub, K., Density functional theory and phytochemical study of Pistagremic acid. Spectrochim Acta A Mol Biomol Spectrosc 2014, 118, 210-4.
56. Yahyaei, H.; Shahab, S.; Sheikhi, M.; Filippovich, L.; Almodarresiyeh, H. A.; Kumar, R.; Dikusar, E.; Borzehandani, M. Y.; Alnajjar, R., Anisotropy (optical, electrical and thermal conductivity) in thin polarizing films for UV/Vis regions of spectrum: Experimental and theoretical investigations. Spectrochim Acta A Mol Biomol Spectrosc 2018, 192, 343-360.
57. Kuzu, B.; Menges, N., Indole-containing new types of dyes and their UV-vis and NMR spectra and electronic structures: Experimental and theoretical study. Spectrochim Acta A Mol Biomol Spectrosc 2016, 162, 61-8.
58. Grante, I., Actins, A.; Orola, L., Protonation effects on the UV/Vis absorption spectra of imatinib: a theoretical and experimental study. Spectrochim Acta A Mol Biomol Spectrosc 2014, 129, 326-32.
59. Atac, A.; Karaca, C.; Gunnaz, S.; Karabacak, M., Vibrational (FT-IR and FT-Raman), electronic (UV-Vis), NMR (1H and 13C) spectra and reactivity analyses of 4,5-dimethyl-o-phenylenediamine. Spectrochim Acta A Mol Biomol Spectrosc 2014, 130, 516-25
60. Almandoz, M. C.; Sancho, M. I., Duchowicz, P. R.; Blanco, S. E., UV-Vis spectroscopic study and DFT calculation on the solvent effect of trimethoprim in neat solvents and aqueous mixtures. Spectrochim Acta A Mol Biomol Spectrosc 2014, 129, 52-60.
61. Sas, E. B.; Kose, E.; Kurt, M.; Karabacak, M., FT-IR, FT-Raman, NMR and UV-Vis spectra and DFT calculations of 5-bromo-2-ethoxyphenylboronic acid (monomer and dimer structures). Spectrochim Acta A Mol Biomol Spectrosc 2015, 137, 1315-33.
62. Abbas, A.; Gokce, H.; Bahceli, S., Spectroscopic (vibrational, NMR and UV-vis.) and quantum chemical investigations on 4-hexyloxy-3-methoxybenzaldehyde. Spectrochim Acta A Mol Biomol Spectrosc 2016, 152, 596-607.
63. Karabacak, M.; Cinar, Z.; Kurt, M.; Sudha, S.; Sundaraganesan, N., FT-IR, FT-Raman, NMR and UV-vis spectra, vibrational assignments and DFT calculations of 4-butyl benzoic acid. Spectrochim Acta A Mol Biomol Spectrosc 2012, 85 (1), 179-89.
64. Karabacak, M.: Kose, E.; Sas, E. B.; Kurt, M.; Asiri, A. M.; Atac, A., DFT calculations and experimental FT-IR, FT-Raman, NMR, UV-Vis spectral studies of 3-fluorophenylboronic acid. Spectrochim Acta A Mol Biomol Spectrosc 2015, 136 Pt B, 306-20.
65. Gelfand, N.; Freidzon, A.; Vovna, V., Theoretical insights into UV-Vis absorption spectra of difluoroboron beta-diketonates with an extended pi system: An analysis based on DFT and TD-DFT calculations. Spectrochim Acta A Mol Biomol Spectrosc 2019, 216, 161-172.
66. Hidalgo, J. R.; Neske, A.; Iramain, M. A.; Alvarez, P. E.; Bongiorno, P. L.; Brandan, S. A., Experimental isolation and spectroscopic characterization of squamocin acetogenin combining FT-IR, FT-Raman and UV-Vis spectra with DFT calculations. J Mol Struct 2020, 1219, 128610.

It will be understood that various details of the presently disclosed subject matter may be changed without departing from the scope of the presently disclosed subject matter. Furthermore, the foregoing description is for the purpose of illustration only, and not for the purpose of limitation.

1. A system for predicting a spectrum of a target molecule, the system comprising: one or more processors and a memory communicably coupled to the one or more processors and storing: a first module comprising instructions that when executed by the one or more processors cause the one or more processors to receive or generate a descriptor of the target molecule; and a second module including instructions that when executed by the one or more processors cause the one or more processors to apply a trained machine learning model to the descriptor of the target molecule to predict a spectrum of the target molecule, and further wherein the second module includes instructions to provide the predicted spectrum as an electronic output.

2. The system of claim 1, wherein the descriptor of the target molecule is a simplified molecular-input line-entry system (SMILES) sequence, a tokenized SMILES sequence, or an extended connectivity fingerprint (ECFP).

3. The system of claim 1, wherein the descriptor of the target molecule is an ECFP and the first module comprises instructions that when executed by the one or more processors cause the one or more processors to divide the ECFP into a plurality of groups, optionally 8 groups, and to convert each of the groups into a decimal value for input into the trained machine learning model.

4. The system of claim 1, wherein the descriptor of the target molecule is a tokenized SMILES sequence and the second module further comprises instructions that when executed by the one or more processors cause the one or more processors to generate a vector of weights for each character of the tokenized SMILES sequence at one or more wavelength values.

5. The system of claim 1, wherein the trained machine learning model is a trained model for time series data prediction and/or a trained long-short term memory (LSTM) model or a machine learning model similar thereto.

6. The system of claim 1, wherein the predicted spectrum is an ultraviolet-visible (UV-Vis) spectrum.

7. The system of claim 1, wherein the system is further configured to receive data related to an observed spectrum of the target molecule and the second module further comprises instructions for comparing the predicted spectrum with the observed spectrum, optionally using Dynamic Time Warping (DTW).

8. The system of claim 1, wherein the second module further comprises instructions for measuring the root-mean-squared deviation (RMSD) of the predicted spectrum.

9. The system of claim 1, wherein the system is further configured to generate the trained machine learning model by:
acquiring or generating training data, wherein said training data comprises (a) a plurality of observed spectra, wherein each of the plurality of observed spectra is the observed spectra for a different training molecule in a training set comprising a plurality of training molecules; and (b) a plurality of descriptors, wherein each of the plurality of descriptors is a descriptor for a different training molecule in the training set; and
training a machine learning model using the training data, thereby generating the trained machine learning model.

10. A method for predicting a spectrum of a target molecule, optionally a UV-Vis spectrum, comprising:
(i) receiving and/or generating a descriptor of the target molecule; and
(ii) applying a trained machine learning model to the descriptor of the target molecule with at least one processor to provide a predicted spectrum of the target molecule.

11. The method of claim 10, wherein the descriptor of the target molecule is a simplified molecular-input line-entry system (SMILES) sequence, a tokenized SMILES sequence, or an extended connectivity fingerprint (ECFP).

12. The method of claim 11, wherein the receiving and/or generating data defining the descriptor of the target molecule of step (i) comprises generating an ECFP of the target molecule and further comprises dividing the ECFP into a plurality of groups, optionally 8 groups, and converting each group into a decimal value.

13. The method of claim 11, wherein the descriptor of the target molecule is a tokenized SMILES sequence and wherein the applying of step (ii) comprises generating a vector of weights for each character in the tokenized SMILES sequence at one or more wavelength values.

14. The method of claim 10, wherein the trained machine learning model is an trained machine learning model for time series data prediction and/or a trained long-short term memory (LSTM) model or a machine learning model similar thereto.

15. The method of claim 10, further comprising comparing the predicted spectrum with an experimentally observed spectrum, optionally using Dynamic Time Warping (DTW).

16. The method of claim 10, further comprising analyzing the predicted spectrum to predict phototoxicity of the target molecule.

17. The method of claim 10, wherein the target molecule is a potential dye or colorant molecule and the predicted spectrum is a visible spectrum that provides information regarding color of the target molecule.

18. The method of claim 10, wherein the method further comprises, prior to step (ii), generating the trained machine learning model, wherein generating the trained machine learning model comprises:
acquiring or generating training data, wherein said training data comprises (a) a plurality of observed spectra, wherein each of the plurality of observed spectra is the observed spectra for a different training molecule in a training set comprising a plurality of training molecules; and (b) a plurality of descriptors, wherein each of the plurality of descriptors is a descriptor for a different training molecule in the training set; and
training a machine learning model using the training data, thereby generating the trained machine learning model.

19. A method for detecting a target molecule in a mixture of molecules, wherein the method comprises: (a) obtaining a spectrum of the mixture of molecules, optionally wherein the spectrum is a UV-Vis spectrum; and (b) comparing the spectrum from (a) with a predicted spectrum of the target molecule, optionally a predicted UV-Vis spectrum of the target molecule, wherein said predicted spectrum is obtained using a system of claim 1.

20. The method of claim 19, wherein the spectrum obtained in step (a) is a spectrum obtained from an aliquot of a chromatography eluant, optionally of a synthetic reaction mixture, further optionally wherein the chromatography eluant is a high-performance liquid chromatography (HPLC) eluant.

21. The method of claim 19, wherein the spectrum obtained in step (a) is a spectrum obtained from a sample present in a well of a microarray or microwell plate, optionally wherein said microarray or microwell plate comprises a plurality of wells and wherein each of the plurality of wells contains a sample that is different from the sample present in any other of the plurality of wells.

22. A method for detecting a target molecule in a mixture of molecules, wherein the method comprises:
(a) providing a microarray or microwell plate comprising a plurality of wells, wherein each of the plurality of wells contains a sample that comprises one or more molecules, and wherein each of the plurality of wells contains a sample that comprises a different molecule or combination of molecules than in a sample present in any other of the plurality of wells;
(b) obtaining a spectrum, optionally a UV-Vis spectrum, of the sample present in each of a plurality of wells of the microarray or microwell plate, thereby obtaining a plurality of spectra; and
(c) comparing the spectra from (b) with a predicted spectrum of the target molecule, optionally a predicted UV-Vis spectrum of the target molecule, wherein said predicted spectrum is obtained using a system of claim 1.

23. A non-transitory computer readable medium comprising computer executable instructions embodied in a computer readable medium that when executed by a processor of a computer control the computer to perform steps comprising:
receiving and/or generating a descriptor of the target molecule, optionally a simplified molecular-input line-entry system (SMILES) sequence, a tokenized SMILES sequence, or an extended connectivity fingerprint of the target molecule; and
applying a trained machine learning model to the descriptor to predict a spectrum of the target molecule.

24. A method for detecting a target molecule in a mixture of molecules, wherein the method comprises: (a) obtaining a spectrum of the mixture of molecules, optionally wherein the spectrum is a UV-Vis spectrum; and (b) comparing the spectrum from (a) with a predicted spectrum of the target molecule, optionally a predicted UV-Vis spectrum of the target molecule, wherein said predicted spectrum is obtained by:
(i) receiving and/or generating a descriptor of the target molecule; and
(ii) applying a trained machine learning model to the descriptor of the target molecule with at least one processor to provide a predicted spectrum of the target molecule.

25. The method of claim 24, wherein the spectrum obtained in step (a) is a spectrum obtained from an aliquot of a chromatography eluant, optionally of a synthetic reaction mixture, further optionally wherein the chromatography eluant is a high-performance liquid chromatography (HPLC) eluant.

26. The method of claim 24, wherein the spectrum obtained in step (a) is a spectrum obtained from a sample present in a well of a microarray or microwell plate, optionally wherein said microarray or microwell plate comprises a plurality of wells and wherein each of the plurality of wells contains a sample that is different from the sample present in any other of the plurality of wells.

27. A method for detecting a target molecule in a mixture of molecules, wherein the method comprises:
(a) providing a microarray or microwell plate comprising a plurality of wells, wherein each of the plurality of wells contains a sample that comprises one or more molecules, and wherein each of the plurality of wells contains a sample that comprises a different molecule or combination of molecules than in a sample present in any other of the plurality of wells;
(b) obtaining a spectrum, optionally a UV-Vis spectrum, of the sample present in each of a plurality of wells of the microarray or microwell plate, thereby obtaining a plurality of spectra; and
(c) comparing the spectra from (b) with a predicted spectrum of the target molecule, optionally a predicted UV-Vis spectrum of the target molecule, wherein said predicted spectrum is obtained by:
(i) receiving and/or generating a descriptor of the target molecule; and
(ii) applying a trained machine learning model to the descriptor of the target molecule with at least one processor to provide a predicted spectrum of the target molecule.
